(19) United States
(12) Patent Application Publication
(10) Pub. No.: US 2026/0335059 A1
(43) Pub. Date: Oct. 29, 2026

(54) SYSTEMS AND DEVICES FOR TRANSPHASIC MOLDING OF MEDICAMENT CARRIER WITH RELEASE POINTS AND METHODS THEREOF

(71) Applicant: 3i Biotech, LLC, Lubbock, TX (US)

(72) Inventors: Rohan Ingrole, Lubbock, TX (US); David Syn, Lubbock, TX (US)

(73) Assignee: 3i Biotech, LLC, Lubbock, TX (US)

(21) Appl. No.: 19/545,013

(22) Filed: Feb. 20, 2026

Related U.S. Application Data
(60) Provisional application No. 63/761,157, filed on Feb. 20, 2025.

(51) Int. Cl. A61C 19/06

(52) U.S. Cl. CPC A61C 19/063

(57) ABSTRACT

A medicament carrier system for delivering therapeutic agents into a gingival crevice proximate to a junctional epithelium. The system includes a carrier frame defining a geometric well configured to receive medicament using transphasic molding, wherein a medicament solution wets the well and transitions to a retained medicament seed as solvent dissipates. The carrier frame may be planar or semi-rigid with three-dimensional depth and may include one or more compartments defined by partitioners to enable phased or multi-drug delivery. The combined thickness of the carrier frame and retained medicament may be less than 1 mm, and preferably less than 600 microns, to permit insertion to the base of the gingival sulcus. A releasable interface couples the carrier to a handle or cartridge, allowing detachment after positioning within the gingival crevice. The carrier may be dissolvable or removable following medicament release.

### CROSS-REFERENCE TO RELATED APPLICATIONS

This patent application claims priority to and the benefit of U.S. Provisional Patent Application No. 63/761,157, filed Feb. 20, 2025, titled “SYSTEMS AND DEVICES FOR TRANSPHASIC MOLDING OF MEDICAMENT CARRIER WITH RELEASE POINTS AND METHODS THEREOF”, the entirety of which is hereby incorporated by reference for all purposes.

### TECHNICAL FIELD

The present disclosure relates generally to medicament delivery devices, and more particularly to devices and methods for transphasic molding of medicament carriers with release points for delivery of medicament to the gingival crevice.

### BACKGROUND

Micronutrient malnutrition is a pervasive global health challenge affecting billions of people worldwide. Essential vitamins and minerals, collectively known as micronutrients, play crucial roles in various physiological processes and must be obtained through dietary intake, as they are not endogenously produced in the body (with the notable exception of vitamin D). Deficiencies in these vital micronutrients can lead to a range of serious health issues, including anemia, compromised immune function, and impaired cognitive development. Additionally, micronutrient deficiencies may contribute to stunted growth in children, increased susceptibility to infections, and poor wound healing. In some cases, these deficiencies can have long-lasting effects on overall health and quality of life.

Certain populations face an elevated risk of micronutrient deficiencies due to specific physiological factors. For example, individuals with obesity are more susceptible to these deficiencies as a result of mechanisms such as fat sequestration, inflammation-associated malabsorption in the gastrointestinal tract, and hyperinsulinemia-associated urinary excretion. This risk is further amplified in patients who have undergone bariatric surgery, as these procedures can significantly alter the anatomy and function of the gastrointestinal tract, leading to reduced micronutrient absorption. Other high-risk groups may include pregnant women, elderly individuals, and those with chronic diseases such as celiac disease or inflammatory bowel disorders. In some cases, dietary restrictions, whether due to personal choice or medical necessity, may also contribute to an increased risk of micronutrient deficiencies.

The conventional approach to addressing micronutrient deficiencies in bariatric surgery patients typically involves a demanding regimen of multiple pills taken several times throughout the day. This method, while potentially effective, places a considerable burden on patients and can lead to decreased adherence over time, potentially compromising the effectiveness of the supplementation strategy. In response to these challenges, alternative methods of micronutrient delivery have been explored, including transdermal patches, subcutaneous or intramuscular injections, nasal sprays, and sublingual applications. However, each of these approaches comes with its own set of limitations, whether in terms of efficacy, cost, or the range of micronutrients that can be effectively delivered. For example, transdermal patches may be limited in the types and quantities of micronutrients they can deliver, while injections may be invasive and require frequent administration by healthcare professionals.

The quest for more effective and user-friendly methods of micronutrient delivery has led to the investigation of various approaches. However, many of these methods face significant challenges in terms of effectively depositing micronutrients in specific target areas, such as gingival crevice. For example, these methods often are limited in the amount and type of medicaments that can be carried by the device that is used to deposit the medicament into the gingival crevice. Even more, techniques for depositing the medicament into the gingival crevice conveniently and efficiently are very limited.

### BRIEF SUMMARY

The present disclosure achieves technical advantages as systems, devices, and methods for transphasic molding of medicament carriers with release points for delivery of medicament to the gingival crevice. Various embodiments described herein provide a medicament carrier configured to carry a medicament within a geometric well defined by a carrier frame and to deliver the medicament into a gingival crevice of a patient for absorption through a junctional epithelium. The medicament carrier of embodiments may be configured to receive medicament using a transphasic loading technique, in which a medicament solution is applied to the carrier frame, wets and conforms to the geometry of the carrier well, and thereafter transitions through one or more phases as solvent dissipates, resulting in a molded medicament seed retained within the carrier frame. In this manner, the medicament carrier of embodiments may provide controlled shaping, retention, and positioning of the medicament within the gingival crevice to enable targeted delivery to the junctional epithelium.

In embodiments, the carrier frame may be configured as a linear framework lying substantially within a single plane, or may be configured as a semi-rigid structure having defined three-dimensional depth. The linear framework configuration may define a medicament well having a thickness substantially corresponding to the thickness of a filament forming the carrier frame. In embodiments, the semi-rigid configuration may define a volumetric well having depth up to approximately 700 microns, allowing increased medicament capacity. In embodiments, the combined thickness of the carrier frame and the medicament retained therewithin may be configured to be less than 1 mm, and preferably less than 600 microns, to permit insertion of the carrier frame to a base of the gingival sulcus proximate to the junctional epithelium to target the junctional epithelium for medicament absorption.

In embodiments, the carrier frame may be configured with a plurality of compartments defined by one or more partitioners. The compartments may be arranged in linear, parallel, diagonal, cross-member, X-shaped, mesh, honeycomb, or other geometric configurations. Each compartment may be independently loaded with medicament during transphasic molding and may be configured to release medicament at the same stage or at different stages following deposition within the gingival crevice. In embodiments, the compartments may contain identical medicament compositions or different medicament compositions, including pure drug, drug combined with excipients, drug encapsulated within nanoparticles, or combinations thereof. In embodiments, the geometry of the carrier frame may operate to define both spatial distribution and temporal sequencing of medicament release.

In embodiments, the material composition of the carrier frame may include monofilament, braided filament, semi-rigid polymer, molded polymer, stamped polymer, composite material, plastic material, metal material, biodegradable material, or combinations thereof. In embodiments, the carrier frame may be formed from a dissolvable material configured to dissolve within the gingival crevice after deposition, or may be non-dissolvable and removable after medicament delivery. In embodiments, the carrier frame material may be infused with medicament in addition to the medicament retained within the well.

In embodiments, the medicament carrier may be configured as part of a system including a reusable handle and a release interface configured to releasably couple the carrier to the handle. The release interface may include releasable inserts, tabs, cylindrical inserts, cartridge inserts, channels and guides, insertion rods and holes, or combinations thereof. The release interface may be configured to enable insertion of the medicament carrier into the gingival crevice using the handle and subsequent detachment of the handle from the medicament carrier after the medicament carrier has been positioned proximate to the junctional epithelium. In embodiments, the medicament carrier may be implemented as part of a removable carrier cartridge configured to be attached to and detached from a reusable handle, thereby enabling repeated medicament applications using interchangeable cartridges.

In embodiments, after insertion of the medicament carrier into the gingival crevice and detachment of the handle via the release interface, the medicament carrier may remain within the gingival crevice for a period sufficient to allow release of the medicament for absorption through the junctional epithelium. In embodiments, the medicament carrier may be left in situ to dissolve along with the medicament, or may be removed after medicament delivery depending on configuration. By integrating transphasic loading, compartmentalized carrier geometries, controlled dimensional constraints, and releasable cartridge systems, the present disclosure provides a modular and adaptable platform for targeted medicament delivery into the gingival crevice and enhanced therapeutic absorption through the junctional epithelium.

It is an object of the disclosure to provide a medicament carrier cartridge configured for delivering a therapeutic agent into a junctional epithelium of a gingival crevice of a patient. It is a further object of the disclosure to provide a system for delivering medicament into a junctional epithelium of a gingival crevice of a patient, and a method of targeting delivery of a medicament to the junctional epithelium of a gingival crevice of a patient using a medicament delivery device.

In one particular embodiment, a medicament carrier cartridge configured for delivering a therapeutic agent into a junctional epithelium of a gingival crevice of a patient is provided. The medicament carrier cartridge includes a structural frame defining a geometric well having a defined depth, the geometric well configured to carry a medicament molded therewithin, and a release interface integrated with the structural frame. In embodiments, the release interface includes one or more release inserts configured to releasably couple the structural frame to a handle device and to facilitate the detachment of the structural frame from the handle device for deposition within the gingival crevice.

In another embodiment, a system for delivering medicament into a junctional epithelium of a gingival crevice of a patient is provided. The system includes a handle device configured to facilitate positioning within an oral cavity of the patient, a medicament carrier including a frame structure carrying a medicament seed and a connection feature configured to interface with the handle device, and a coupling mechanism formed between the handle device and the medicament carrier. In embodiments, the coupling mechanism includes one or more detachable release points configured to mechanically connect the medicament carrier to the handle device during insertion and to separate the medicament carrier from the handle device upon deposition of the medicament seed into the gingival crevice for absorption of the medicament through the junctional epithelium.

In yet another embodiment, a method of targeting delivery of a medicament to the junctional epithelium of a gingival crevice of a patient using a medicament delivery device configured is provided. The method includes positioning a medicament carrier frame of a medicament delivery device proximate to a gingival crevice of a patient using a handle. In embodiments, the medicament carrier frame is releasably coupled to a handle of the medicament carrier device, and the medicament carrier is configured to carry a medicament. The method also includes inserting the medicament carrier frame into the gingival crevice until the medicament carrier frame reaches a base of the gingival crevice proximate to a junctional epithelium, detaching the medicament carrier frame from the handle by separating a release interface connecting the medicament carrier frame to the handle, and retaining the detached medicament carrier frame within the base of the gingival crevice for a period sufficient for the medicament to be absorbed through the junctional epithelium.

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings, in which:

FIG. 1A shows a perspective view of a portion of an oral cavity illustrating a representative gingival crevice adjacent to a tooth.

FIGS. 1B-1E show exemplary anatomy of a gingival crevice in the gingiva for targeting deposition of medicament in accordance with embodiments of the present disclosure.

FIGS. 1F-1H shows various examples of medicament seed insertion into the gingival crevice in accordance with embodiments of the present disclosure.

FIG. 1I illustrates a front view of the gingival crevice anatomy of a patient that may be targeted for medicament deposition in accordance with embodiments of the present disclosure.

FIGS. 2A–2C illustrate examples of a medicament carrier configured to carry medicament for deposition into a gingival crevice of a patient for absorption through the junctional epithelium in accordance with embodiments of the present disclosure.

FIG. 2D illustrates an example of a carrier frame that is configured with a three-dimensional geometry defining a depth 255 in accordance with embodiments of the present disclosure.

FIG. 3 illustrates an example system representing an implementation of a linear framework configuration for delivering medicament into a gingival crevice of a patient for absorption through the junctional epithelium in accordance with embodiments of the present disclosure.

FIGS. 4A–4C illustrate examples of a two-compartment linear framework configuration implemented using a carrier arm in accordance with embodiments of the present disclosure.

FIGS. 5A–5C illustrate an example of a three-compartment linear framework configuration implemented using a carrier arm in accordance with embodiments of the present disclosure.

FIGS. 6A–6C illustrate embodiments of a four-compartment linear framework configuration implemented using a carrier arm in accordance with embodiments of the present disclosure.

FIG. 7A illustrates various example geometries of a semi-rigid carrier frame configured in accordance with embodiments of the present disclosure.

FIG. 7B illustrates the example geometries of the carrier frame after loading with medicament in accordance with embodiments of the present disclosure.

FIG. 8A illustrates examples of a semi-rigid carrier arm configured with defined three-dimensional depth and internal patterned geometries in accordance with embodiments of the present disclosure.

FIG. 8B further illustrates an example of the carrier arm after loading with medicament in accordance with embodiments of the present disclosure.

FIG. 9 illustrates an exemplary system configured to provide releasable coupling between a carrier arm and a handle via a release interface in accordance with embodiments of the present disclosure.

FIGS. 10A and 10B illustrate examples of a coated carrier arm configured in accordance with embodiments of the present disclosure.

FIGS. 11A and 11B illustrate examples of a release interface configured to releasably couple a carrier frame to a handle in accordance with embodiments of the present disclosure.

FIGS. 12A–12C illustrate examples of a release interface configured to releasably couple a carrier frame to a handle in accordance with embodiments of the present disclosure.

FIG. 13 illustrates an exemplary system in which the carrier is implemented as part of a carrier cartridge in accordance with embodiments of the present disclosure.

FIG. 14 illustrates an example of the cartridge-based release interface in which cartridge inserts are used to releasably attach a carrier cartridge to a reusable handle in accordance with embodiments of the present disclosure.

FIG. 15 illustrates an exemplary system in which the head of the carrier cartridge is configured as part of a removable cartridge assembly that is attachable to and detachable from a reusable handle in accordance with embodiments of the present disclosure.

FIG. 16 illustrates an embodiment of a system in which the carrier cartridge includes a head that is removably attachable to a reusable handle using insertion rods and holes configured as cartridge inserts in accordance with embodiments of the present disclosure.

FIG. 17 shows a high-level flow diagram of operations for a medicament delivery device including a releasable carrier frame configured to carry a medicament seed and including one or more releasable points enabling deposition within the gingival crevice for absorption through the junctional epithelium in accordance with embodiments of the present disclosure.

It should be understood that the drawings are not necessarily to scale and that the disclosed embodiments are sometimes illustrated diagrammatically and in partial views. In certain instances, details which are not necessary for an understanding of the disclosed methods and apparatuses, or which render other details difficult to perceive may have been omitted. It should be understood, of course, that this disclosure is not limited to the particular embodiments illustrated herein.

### DETAILED DESCRIPTION

The disclosure presented in the following written description and the various features and advantageous details thereof, are explained more fully with reference to the non-limiting examples included in the accompanying drawings and as detailed in the description. Descriptions of well-known components have been omitted to not unnecessarily obscure the principal features de-scribed herein. The examples used in the following description are intended to facilitate an understanding of the ways in which the disclosure can be implemented and practiced. A person of ordinary skill in the art would read this disclosure to mean that any suitable combination of the functionality or exemplary embodiments below could be combined to achieve the subject matter claimed. The disclosure includes either a representative number of species falling within the scope of the genus or structural features common to the members of the genus so that one of ordinary skill in the art can recognize the members of the genus. Accordingly, these examples should not be construed as limiting the scope of the claims.

A person of ordinary skill in the art would understand that any system claims presented herein encompass all of the elements and limitations disclosed therein, and as such, require that each system claim be viewed as a whole. Any reasonably foreseeable items functionally related to the claims are also relevant. The Examiner, after having obtained a thorough understanding of the disclosure and claims of the present application has searched the prior art as disclosed in patents and other published documents, i.e., nonpatent literature. Therefore, the issuance of this patent is evidence that: the elements and limitations presented in the claims are enabled by the specification and drawings, the issued claims are directed toward patent-eligible subject matter, and the prior art fails to disclose or teach the claims as a whole, such that the issued claims of this patent are patentable under the applicable laws and rules of this country.

It is noted that the oral cavity contains multiple gingival crevices, each providing an advantageous site for depositing medicament seeds that can dissolve and release their medicaments. By targeting these gingival crevices, and particularly the junctional epithelium located deep within them, the present disclosure leverages the high permeability of this anatomical region and its close proximity to connective tissue populated by immune cells. This approach not only improves bioavailability but also has the potential to reduce systemic side effects commonly associated with oral or injectable medicament delivery.

The inventors have found that the field of medicament delivery has seen a variety of routes, methods and devices developed over the years. The inventors have also found that these methods and devices may be employed to deliver a range of medicaments, including but not limited to nutrients, micronutrients, antibiotics, antigens, anti- inflammatory agents, antimicrobial agents, antibodies, steroids, DNA, and minerals. The delivery of these medicaments may be targeted to specific areas of the body to maximize their efficacy and minimize potential side effects. Additionally, the route of medicament delivery can have a significant impact on its pharmacokinetics and pharmacodynamics.

The route of delivery and the characteristics of the medicament itself, in its various forms may be factors that determine the amount that can be delivered, bioavailability, biodistribution, and the duration of circulation within the body. These factors are important in determining the choice of route of delivery for a given medicament. However, other concerns such as safety, efficacy, ease of administration, whether self-administered or given by a health care professional, costs, ease of manufacturing and stability of the medicament in the form determined by the route, also play significant roles. Matching the individual characteristics of the medicament to be delivered, in its various forms, to the routes through which it can be delivered, to accomplish the highest safety, efficacy, ease of administration, reduction of costs, and ease of manufacturing is a significant goal in developing novel medicament delivery systems. A novel device and route to deliver medicament to a space that has not been previously used for medicament delivery, the Gingival Crevice (GC), able to overcome many of the limitations of the commonly known routes of delivery, is described herein.

The classification of routes for administration of medicament is usually determined by the location of application. The two most commons routes of medicament delivery are broadly categorized as enteral (through the gastrointestinal tract), and parenteral (not enteral). The enteral route involves absorption of the medicament through the gastrointestinal (GI) tract. Administration can be through ingestion or other access to the GI tract such as tubes or catheters or insertion into the rectum. The parenteral route refers to any route that is not enteral. Parenteral administration can involve injection of medicament via a needle and syringe, or by insertion of an indwelling catheter into a body cavity, or by application of medicament on a surface of the body. Parenteral routes are often described with prefixes such as epi- (epidural), sub- (subcutaneous), intra- (intramuscular), extra- (extra-amniotic), peri- (perivascular) and trans- (transdermal or transmucosal) depending on where the medicament is deposited and how it is being taken up by the body.

The inventors have found that the enteral route is advantageous in that it is available for most individuals, is convenient, self-administrable, allows for systemic distribution and is often cost-effective. The disadvantages of the enteral route include the higher dosage of medicament required due to the barriers inherent to the GI tract. These barriers include, but are not limited to, the degradative acidic environment of the stomach, the first-pass effect (reductive filtration, enzymatic, and proteolytic process enacted through the liver during digestive processes resulting in significant removal of administered drug and thus reduced bioavailability for systemic circulation), and interactions with other medicaments. These disadvantages are magnified in situations where absorption has been surgically altered at the same time requirements for micronutrients are increased such as in the bariatric surgical population, or in situations where the enteral route is not available.

The inventors have found that the parenteral route is advantageous in that it can be rapid, can be delivered directly into the blood stream, can avoid the first-pass effect, can deliver much higher amounts of medicaments, can be utilized when the enteral route is not available and can be easily applied topically. The disadvantages of the parenteral route are that it can be painful, inconvenient, expensive, locally irritating, relatively difficult to store in some forms and may require administration by a health care professional. Both the enteral and parenteral routes of medicament delivery have limitations.

The GC, also known as the Gingival Sulcus, is a unique space that has special characteristics dictated by the environment it is in. The nature and the location of the GC requires it to have unique physical and biological characteristics as it is the barrier to the external environment and is under constant assault requiring it to be highly immunologically active. To facilitate this immune surveillance, the specialized mucosa of the GC is highly permeable allowing for rapid cellular motility and transport. The inventors have found that these unique characteristics make the GC an ideal target for immunomodulation. In addition, the high permeability of the GC also makes it an ideal site for medicament delivery, overcoming some of the limitations of both the enteral and commonly known parenteral routes. A novel device to deliver medicament to the GC, such as the devices described herein, can take advantage of these unique characteristics.

The GC is a dynamic fluid filled moat which is the interface between a tooth and the surrounding gingival tissue. It is a potential space filled with food debris, endogenous and exogenous cells, and chemicals. Any individual that has flossed is familiar with the characteristics of this potential space. In the deepest recesses of the GC is the Junctional Epithelium (JE) tissue which is a stratified squamous epithelium that resembles the oral epithelium but differs in some important ways. It is the only discontinuous epithelium layer in the body. The JE is the final barrier between what is inside the body and what is outside. Unlike the rest of the oral mucosa, cells of the JE are non-keratinized, have a high turnover rate, and have wide intercellular spaces which gives the JE its characteristic high permeability. The inventors have found that this high permeability of the JE makes it susceptible as a pathway for metabolic products of plaque bacteria, however this high permeability also makes it an ideal site for rapid absorption of medicament that are delivered to the GC. As the GC is the interface between the inside of the body and the outside world it is a highly immunological space. The JE expresses several cytokines and chemokines and is penetrated by neutrophil and Langerhans cells (specialized dendritic cells). The JE plays a key role in the innate immune system and in the overall health of gingival mucosa. The JE located at the interface between the gums and tooth surface, is a mucosal site. Additionally, the underlying connective tissue of the JE is vascularized. The vascularization of the underlying tissue helps to support immune responses and tissue repair in the gingiva. This vascularization enhances the JE’s immune response by allowing immune cells to be recruited quickly during infection. This biological nature of the JE is crucial for maintaining its barrier function, immune defense, and periodontal health. The inventors have found that, as an active mucosal site and providing access to blood vessels in the underlying connective tissue, the JE can provide an advantage of inducing both mucosal and systemic responses, which makes the GC, specifically the JE of the GC, an ideal space for immunomodulation.

There are currently no methods for accessing the GC to deliver medicament to the deepest recesses to exploit the immune and absorptive potential of the junctional epithelium in the gingival crevice. A novel device designed to accurately target the deep GC, such as the devices and methods described herein, may take advantage of the unique characteristics of this space to safely, efficiently, consistently, quantifiably, painlessly and effectively deliver medicament through a novel route for systemic circulation and for immunomodulation.

This novel route of medicament delivery has not been previously described. It would fall under the general category of parenteral route of delivery. Specifically, such a route for medicament delivery may be characterized as a transmucosal route of delivery (diffusion through a mucosal membrane). Examples of transmucosal routes include insufflation (snorting of cocaine), sublingual (under the tongue) and sublabial (between the lips and gingiva), buccal (inner cheeks), and rectal (mucosal absorption but considered enteral as it is absorbed through the gastrointestinal tract). The JE of the gingival crevice, however, deserves special attention in that it is being utilized, in the present disclosure, for medicament delivery in a novel previously undescribed manner with a previously undescribed device. Unlike other transmucosal routes of delivery, the medicament is not placed into an open space, it is being wedged into a potential space that requires a novel device to deliver it. This potential space is the gingival crevice. The Merriam-Webster dictionary defines crevicular as: “of, relating to, or involving a crevice and especially the gingival crevice.” As such, the inventors have coined the phrase “Intra-crevicular route”. The route of delivery is thus referred to as the “INTRACREVICULAR ROUTE” of delivery. As the Junctional Epithelium is also unique and is the only epithelium in the body that is discontinuous, the route can also be referred to as the “TRANS-JUNCTIONAL ROUTE” and the delivery of medicaments can be appropriately referred to as “TRANS-JUNCTIONAL DELIVERY”.

The proposed novel route of junctional epithelium of the gingival crevice is unique and can be utilized to deliver an array of medicaments each with its unique therapeutic utility. The scope of some of the medicaments, their current challenges in terms of achieving therapeutic effect, and the potential of the JE as an alternative novel route for their delivery are discussed herein. Vaccines play a crucial role in protecting public health by preventing the spread of infectious diseases. They stimulate the immune system to recognize and fight specific pathogens, without causing the disease itself. This not only helps individuals build immunity but also reduces the overall prevalence of diseases, contributing to herd immunity. Vaccines have led to the eradication or significant reduction of deadly diseases like smallpox and polio. They are essential for controlling outbreaks, reducing healthcare costs, and improving life expectancy globally. Conventional vaccine delivery methods, including intramuscular (IM), subcutaneous (SC), and oral routes, have proven effective but come with significant limitations, especially in their ability to induce both systemic and mucosal immune responses. These routes are critical for preventing various infectious diseases, but each has its own set of challenges that affect their overall effectiveness, particularly for certain pathogens. IM and SC routes are commonly used for vaccines, especially for diseases requiring strong systemic immunity, such as hepatitis or tetanus. These methods ensure that the vaccine enters the bloodstream, where a systemic immune response can be triggered. However, the IM and SC routes fail to induce significant mucosal immunity, which is essential for defending against infections at incursion points like the respiratory or gastrointestinal tracts. Most viruses enter the human body through mucosal surfaces, such as the nose, mouth, eyes, and respiratory or gastrointestinal tracts. These sites serve as primary entry points for many pathogens thus it would be advantageous for a delivery route to allow for an induction of an antibody response at these mucosal sites in sync with systemic immune response, so that the attack from viruses can be thwarted at their point of entry. For diseases that primarily affect mucosal surfaces (e.g., influenza or enteric diseases), the conventional routes could be less effective because they might fail to engage the immune systems located in mucosal tissues. Vaccines that are delivered to the JE of the GC could overcome the limitations of conventional vaccine delivery routes and confer both systemic and mucosal immunity.

Micronutrient malnutrition affects over two billion individuals globally, with a particularly acute impact on children under five, more than half of whom suffer from vitamin and mineral deficiencies. Novel methods of micronutrient delivery, such as described herein, may help solve this problem. Micronutrients, which are not endogenously produced in the body—with the exception of vitamin D—must be obtained through dietary intake. In the United States alone, vitamin D deficiency is prevalent in 8% of the population, a figure that is likely much higher on a global scale. Essential minerals such as Iron, Zinc, and Iodine play a central role in preventing anemia, bolstering immune function, and supporting healthy cognitive development in infants. Similarly, Vitamin A is imperative for maintaining healthy eyesight and immune function, preventing blindness, and mitigating the risk of death from infections like measles and diarrhea.

Obese populations are particularly susceptible to micronutrient deficiencies due to factors such as fat sequestration, inflammation-associated malabsorption in the gut, and hyperinsulinemia-associated urinary excretion. These deficiencies are exacerbated with increasing obesity, especially among premenopausal obese females. The prevalence of morbid obesity and the corresponding rise in bariatric surgeries—recognized as the sole effective long-term weight loss treatment—further compound the issue. In 2022, the United States saw 280,000 bariatric procedures, reflecting a growing trend. Post-surgery, individuals experience a marked reduction in micronutrient absorption due to alterations in the stomach and small intestine, necessitating lifelong supplementation of oral vitamins and minerals to prevent deficiencies and associated diseases such as Wernicke’s encephalopathy.

The conventional method of micronutrient supplementation for individuals who have undergone bariatric surgery typically involves the intake of a large number of pills, often exceeding twenty, multiple times throughout the day. This regimen, while effective in theory, presents a considerable burden to the individual. The sheer volume of pills and the frequency of administration can be overwhelming, leading to a decline in adherence over time. This lack of adherence can have serious health implications, as these individuals are already at a heightened risk of micronutrient deficiencies due to the nature of their surgical procedures.

Alternative methods of supplementation have been explored, but these too present their own set of challenges. Transdermal patches, for instance, have been introduced as a less invasive option. However, these patches have not demonstrated the same level of efficacy as oral supplementation. Clinical trials conducted by independent investigators have shown that these patches are considerably less effective than oral supplementation, resulting in higher rates of micronutrient deficiency when used as the sole method of supplementation.

Other forms of supplementation, such as subcutaneous or intramuscular injections, while effective, are not without their drawbacks. These methods can be quite costly, putting them out of reach for many individuals. Additionally, they can be painful, further discouraging adherence to the supplementation regimen.

Nasal sprays and sublingual applications have also been explored as alternative methods of supplementation. While these methods have been shown to be effective, they are limited in their scope. They are typically used for the delivery of specific vitamins, such as vitamin B12, and cannot provide a comprehensive solution for micronutrient supplementation. Furthermore, these methods can be quite expensive, further limiting their accessibility and use.

Other delivery methods, such as medicated floss, have proven to be less than ideal. The primary issue with these methods is that they fail to effectively deposit the micronutrients in the target area, which is the junctional epithelium of the gingival crevice. This is due to the fact that the medicament, or the substance carrying the micronutrients, is often scraped off outside of the gingival crevice during the process of flossing. As a result, a majority of the micronutrients do not reach the intended area and are instead wasted. This inefficiency in delivery not just reduces the effectiveness of the supplementation but also leads to wastage of the medicament. Moreover, these methods are often complex and require a high level of individual compliance for effectiveness. For instance, the use of medicated floss requires the individual to floss regularly and correctly, which can be a challenge for many. This complexity and the associated difficulty in maintaining regular use can lead to poor compliance, further reducing the effectiveness of these methods.

Pharmaceutical medications have greatly influenced health systems by enhancing treatment results and increasing life expectancy. They have changed the management of chronic conditions, including diabetes and hypertension, enabling people to lead healthier lives. Medications have transformed previously deadly conditions into more manageable ones, lowering death rates. Furthermore, the emergence of personalized medicine and targeted therapies has resulted in more efficient treatments, particularly in cancer treatment. Hormonal treatments have significantly changed lives, particularly for those with endocrine issues or experiencing life phases like menopause or hormone therapy post-surgery. For instance, thyroid hormone replacements assist in controlling hypothyroidism, whereas insulin therapy has been crucial for individuals with diabetes. Hormone replacement therapy (HRT) has reduced menopausal symptoms like hot flashes and mood changes, enhancing the quality of life for numerous women. These medications have enabled individuals to live healthier, more active lifestyles, offering relief from severe symptoms and facilitating improved control of chronic illnesses. In the end, pain medications and hormones have allowed individuals to take charge of their health, positively impacting their mental and physical wellness. Nonetheless, the method of drug delivery poses various challenges that can influence treatment efficacy and patient adherence. For instance, oral medications frequently encounter challenges with absorption and bioavailability, as they may be degraded in the digestive tract, diminishing their effectiveness. Injections, although more efficient for direct delivery, may lead to pain, discomfort, and local side effects such as swelling or irritation. Methods such as transdermal patches or sublingual tablets could enhance patient adherence by providing less frequent dosing, but they might not be appropriate for every kind of medication. Reaching controlled and precise drug release continues to pose a challenge, particularly with oral or transdermal techniques, and ensuring stability along with appropriate storage conditions complicates matters further. In addition, guaranteeing accurate drug administration to the designated location, such as in targeted therapies for cancer, remains a significant hurdle. These factors make choosing the right route of administration crucial for successful treatment.

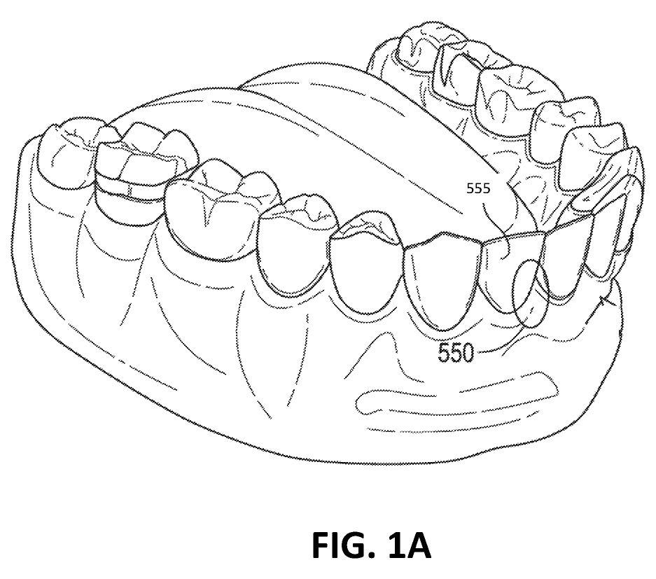

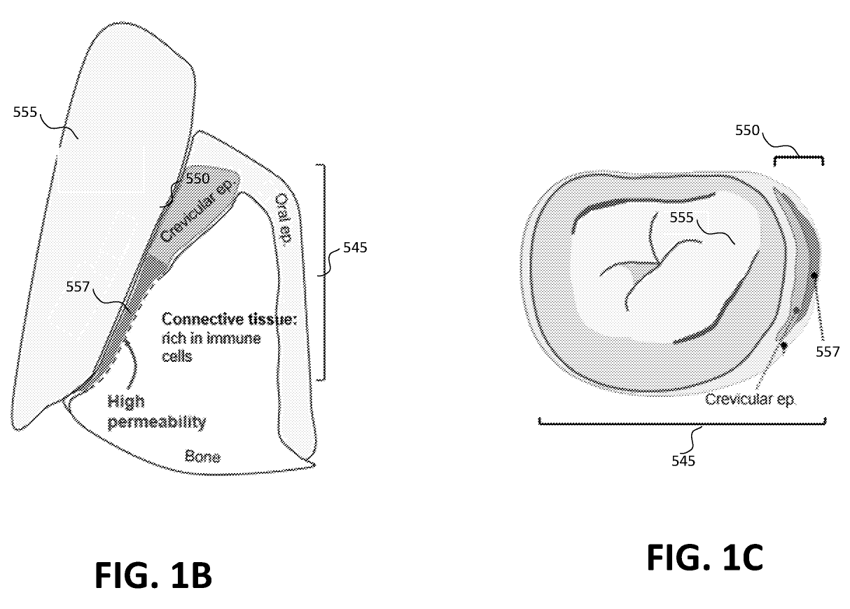

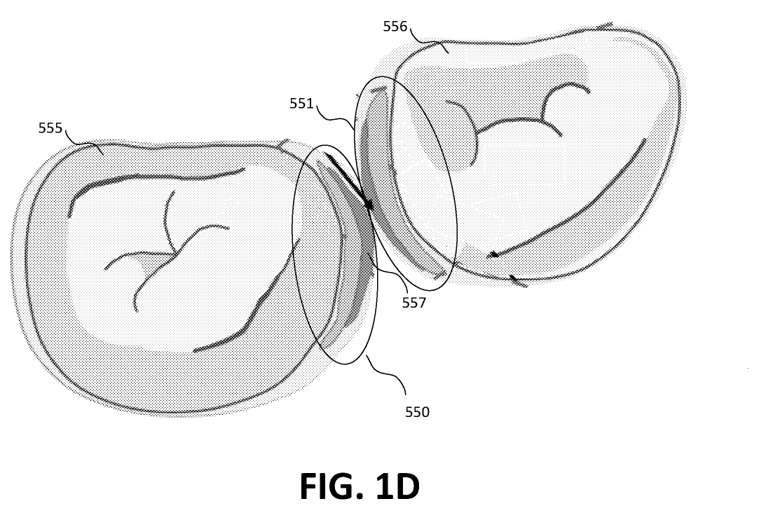

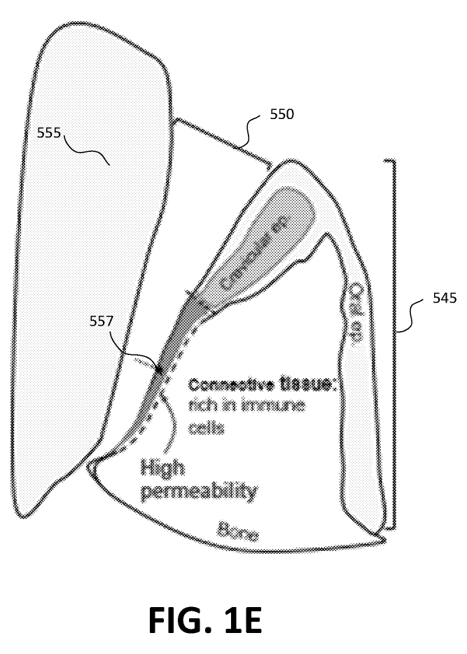

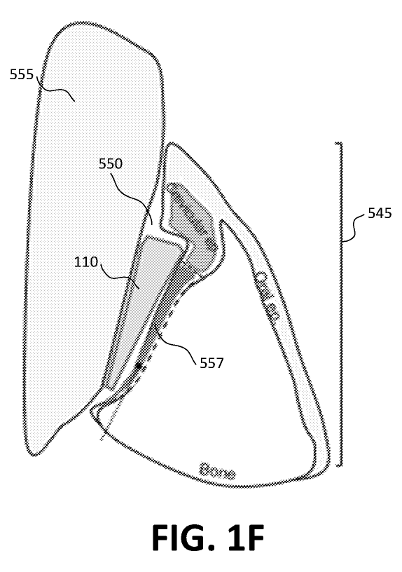

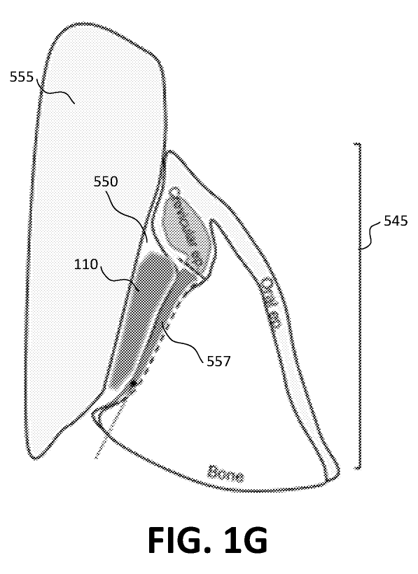

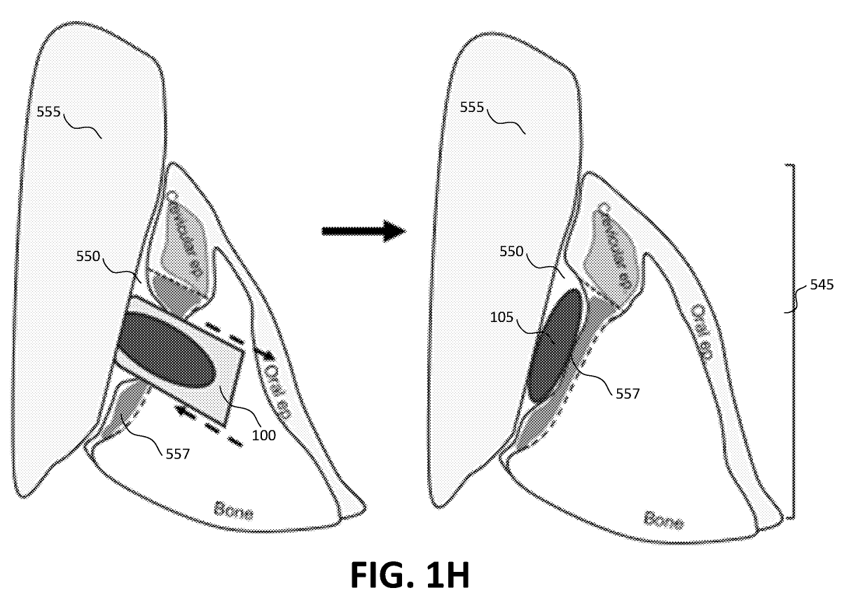

One approach is the delivery of medicaments through the gingival crevice 550, which may include the space between the tooth 555 and the tissue of the gingiva 545. FIG. 1A shows a perspective view of a portion of an oral cavity illustrating a representative gingival crevice 550 adjacent to a tooth 555 under normal conditions. Although FIG. 1A highlights a single tooth and its associated gingival crevice for clarity, it should be recognized that the oral cavity typically includes multiple gingival crevices. Each gingival crevice extends between the tooth surface and the gingival margin, continuing down to the junctional epithelium. Depositing a medicament seed within this space allows the medicament to be released gradually and absorbed rapidly through the specialized epithelial layer of the gingival crevice 550. The anatomical layout and physiological environment of the gingival crevice 550 will be discussed in more detail with reference to FIGS. 1B-1E.

More specifically, the junctional epithelium within the gingival crevice 550 shows considerable promise as a target for medicament delivery. The junctional epithelium has a high permeability due to its unique cellular structure and close contact with the underlying connective tissues, which are rich in immune cells. Because of this anatomical arrangement, the junctional epithelium allows medicaments to be rapidly absorbed directly into systemic circulation. As a result, this route has the potential to be more effective compared to conventional delivery methods.

FIGS. 1B-1E show an exemplary anatomy of a gingival crevice in the gingiva for targeting deposition and enhanced retention of medicament in accordance with embodiments of the present disclosure. It is noted that the illustration of the gingival crevice anatomy in FIGS. 1B-1E is intended to facilitate understanding the target region for medicament delivery as well as the challenges that arise from targeting the gingival crevice and the junctional epithelium for medicament delivery.

As shown, FIGS. 1B-1E illustrate detailed representations of the gingival crevice 550 within the gingiva 545, adjacent to the tooth 555. FIG. 1B shows a sagittal view of a representation of the tooth 555 and gingiva 545 under normal conditions. FIG. 1C shows a caudal (top-down) view that illustrates the overall geometry and orientation of the gingival crevice 550 relative to the tooth 555 structure. In embodiments, the gingival crevice 550 may be characterized as a potential space, which may indicate that although the crevicular epithelium and junctional epithelium 557 are normally in contact with the tooth 555, a space can be created within the gingival crevice 550 by insertion. This region contains two critical epithelial structures which may include the crevicular epithelium lining the crevice surface, which exhibits moderate bioabsorbability for medicament uptake, and the junctional epithelium 557 located deeper within the gingival crevice 550. The junctional epithelium 557 demonstrates high permeability, potentially attributable to its specialized cellular architecture and proximity to connective tissues rich in immune cells, allowing medicaments to permeate and be absorbed into the patient’s system. In particular, the junctional epithelium 557 may include mucosa type tissue that may allow medicament deposited or released within the gingival crevice 550 to “seep through” the junctional epithelium 557 and to be absorbed into the patient’s system. This anatomical arrangement may facilitate rapid, efficient absorption of therapeutic agents into the systemic circulation, which may provide and advantageous path for targeted medicament delivery.

FIG. 1D shows a top view of the exemplary anatomy of the gingival crevice 550 in the gingiva for targeting deposition of medicament in accordance with embodiments of the present disclosure. In particular, FIG. 1D shows two adjacent teeth, 555 and 556, and illustrates the anatomical layout of their corresponding gingival crevices 550 and 551. In this FIG. 1D, tooth 555 is associated with gingival crevice 550, while tooth 556 corresponds to gingival crevice 551. FIG. 1D illustrates the spatial relationship between the two gingival crevices 550 and 551, providing a depiction of how the gingival tissues interface with the tooth structures. In addition, this top-down view not only illustrates the positioning of the teeth 555 and 556 but also highlights the distinct anatomical characteristics of each gingival crevice 550 and 551. As shown, the gingival crevices 555 and 556 may be located at the juncture where the gingival margin meets the tooth surface, defining a narrow space that can be leveraged for the targeted deposition of medicament seeds for absorption through the junctional epithelium.

The inventors have found that achieving effective medicament delivery through the junctional epithelium 557 requires more than simply introducing a medicament into the general vicinity of the gingival crevice 550. For example, merely applying a liquid or fluid formulation into the gingival crevice 550 may be insufficient for facilitating absorption through the junctional epithelium 557. This is partly because of the mucosal nature of the junctional epithelium itself, which means that effective permeation and subsequent systemic uptake may require a certain duration of contact time for the medicament to diffuse across this tissue barrier of the junctional epithelium 557. Fluids, lacking structure, may typically clear quickly from the gingival crevice 550 by natural processes like salivary flow and tissue movement, preventing sustained contact.

In addition, the physical nature of the gingival crevice 550 may present a challenge for fluid-based delivery. For example, the gingival crevice 550 is a potential space, which may mean that the opposing tissues (e.g., tooth surface and gingival epithelium) are normally in contact or close apposition. Creating and maintaining an actual space for medicament placement requires physical displacement or wedging of the tissues. A liquid may lack the physical structure to create the space. For example, a fluid cannot effectively wedge open the potential space upon application, or even prop the space open against the natural tendency of the tissues to return to contact.

In embodiments, the medicament seeds described in various embodiments of the present disclosure may be specifically configured to overcome these limitations. For example, the medicament seeds of embodiments may possess a sufficiently defined physical form (e.g., solid, semi-solid, semi-amorphous) having the structural integrity to interact mechanically with the gingival crevice 550’s environment. Upon insertion, the medicament seed (or the deposition device arm in some embodiments) may operate as a wedge, physically creating a space between the tooth and the gingival tissues for deposition of the medicament seed.

The physical presence of the medicament seed within the gingival crevice 550 may not only create the space but may also maintain it for a duration determined by the medicament seed’s properties (e.g., dissolution rate, etc.). By propping open the gingival crevice 550, the medicament seed may ensure sustained contact between the dissolving carrier, the released medicament, and the junctional epithelium 557. This may allow the medicament to effectively permeate the junctional epithelium 557 for absorption into the patient’s system.

FIG. 1E shows a perspective view of a portion of an oral cavity illustrating a representative gingival crevice 550 adjacent to a tooth 555 under an open condition. For example, FIG. 1E presents an illustrative, somewhat exaggerated representation of the gingival crevice 550 to emphasize the potential space that may be utilized for medicament delivery in accordance with embodiments of the present disclosure. This view represents a scenario where the gingiva 545 is retracted or pulled away from the tooth 555, revealing the space of the gingival crevice 550 between the surface of the tooth 555 and the adjacent epithelial tissues. This visualization is intended to help in understanding the target anatomical space for the deposition of medicament seeds in accordance with embodiments of the present disclosure.

As shown, the created space within the gingival crevice 550 exhibits a generally V-shaped configuration, narrowing towards its apical end. Within this space, distinct regions may be identified relative to the epithelial lining. The upper region of the gingival crevice 550 space may be proximate to the crevicular epithelium, while the lower region may extend deeper, adjacent to the junctional epithelium 557. The junctional epithelium 557, as previously noted, includes characteristics highly favorable for systemic absorption.

The depiction in FIG. 1E underscores the objective of targeted medicament delivery within the gingival crevice 550. In particular, embodiments of the present disclosure may aim to insert a medicament seed such that it is deposited, at least partially, within the lower region of the gingival crevice 550, in close proximity to the junctional epithelium 557. Positioning the medicament seed in this apical area may leverage the high permeability of the junctional epithelium 557. For example, as the medicament seed dissolves and releases its medicament payload, the medicament may be presented to the junctional epithelium 557 for absorption into the patient’s systemic circulation.

FIGS. 1F-1H shows various examples of medicament seed insertion into the gingival crevice in accordance with embodiments of the present disclosure. FIG. 1F shows an exemplary operational scenario following the insertion of a medicament seed 110 into the gingival crevice 550 adjacent to tooth 555. In this example, the medicament seed 110 may include a solid and dissolvable physical structure. As shown, the deposition of the medicament seed 110 into the gingival crevice 550 may physically displace the gingival tissue of the gingiva 545 away from the tooth 555 surface, creating an actual space within the potential gingival crevice 550 in which the medicament seed 110 may reside. The creation and maintenance of the space may be due to the medicament seed 110’s solid form, which may enable it to resist the natural tendency of the gingival tissue to remain in close apposition to the tooth 555.

As also shown, the gingival tissue of the gingiva 545 may inherently seek proximity to the tooth 555, and may conform or wrap around the contours of the solid medicament seed 110. In embodiments, this may result in the upper region of the gingival crevice 550 remaining substantially closed or in contact with the tooth 555, while the medicament seed 110 may occupy the space more significantly in the lower region, particularly proximate to the junctional epithelium 557.

In embodiments, the tendency of the gingival tissue of the gingiva 545 to press against the inserted medicament seed 110 may generates inherent pressure within the gingival crevice 550. This pressure, larger in the deeper V-shaped portion near the junctional epithelium 557, may contribute to forces that may tend to displace or expulse the medicament seed 110 from the crevice 550. It is noted that retention mechanisms to counteract this displacement effect are discussed herein. Notwithstanding this displacement tendency, the placement shown in FIG. 1F positions the dissolvable medicament seed 110 ideally for medicament release. For example, as the medicament seed 110 dissolves over time, the medicament seed 110 may release the medicament directly into the maintained space adjacent to the highly permeable junctional epithelium 557, which may lead to absorption into the patient’s system through the junctional epithelium.

FIG. 1G illustrates another embodiment of the medicament seed 110 positioned within the gingival crevice 550 adjacent to tooth 555. In this example, the medicament seed 110 may be configured with semi-amorphous properties, such as resembling a gel, paste, or other formulation that may not be fully solid nor fully fluid. In this example, the composition of the medicament seed 110 may provide sufficient structural integrity to physically create and maintain a space within the gingival crevice 550 upon deposition.

In embodiments, the semi-amorphous medicament seed 110 shown in FIG. 1G may be able to conform, at least partially, to the contours of the gingival crevice 550. For example, the medicament seed 110 may have enough structure to wedge the potential space open, but flexible enough to adapt to the anatomical geometry of the gingival crevice 550, which may enable the medicament seed 110 to potentially increase the surface area of contact between the medicament seed 110 and the surrounding gingival tissues of the gingiva 545, including the junctional epithelium 557.

In embodiments, despite its ability to conform, the semi-amorphous medicament seed 110 may effectively maintain the space within the gingival crevice 550 to ensure that the released medicament remains localized near the target absorption site. For example, as the medicament seed 110 dissolves or degrades within the gingival crevice 550, the medicament payload of the medicament seed 110 may be released proximate to the junctional epithelium 557 to be absorbed into the patient’s system through the junctional epithelium 557.

FIG. 1H illustrates another embodiment of a medicament seed 105 positioned within the gingival crevice 550 adjacent to tooth 555 using a deposition device 100. In this embodiment, the medicament seed 105 may be formulated as a coating applied to at least a portion of the deposition device 100. In this example, the deposition device 100 carrying the medicament seed coating 105 may be inserted into the gingival crevice 550. The insertion of the device 100 may operate to create a space within the gingival crevice 550between the gingiva 545 and the tooth 555.

Once the deposition device 100 is positioned within the gingival crevice 550, such as at the base or deepest portion of the gingival crevice 550, particularly near the desired target region such as the junctional epithelium 557, the medicament seed 105, which may be a coating, may be transferred from the deposition device 100 into the space of the gingival crevice 550. In embodiments, the transfer may be facilitated by some actions, such as agitating the deposition device 100, moving the deposition device 100 back and forth within the gingival crevice 550, or employing another mechanism configured to release or dislodge the coating (e.g., the medicament seed 105) from the surface of the deposition device 100. FIG. 1H shows both the deposition device 100 within the crevice and the released medicament seed 105 residing therein post-transfer.

In embodiments, following the release of the medicament seed 105 from the deposition device 100, the medicament seed 105, now present as deposited material within the gingival crevice 550, may dissolve and release the embedded or carried medicament into the gingival crevice 550 for absorption through the junctional epithelium 557 and into the patient’s system.

As mentioned above, a primary objective for effective therapy is ensuring the medicament seed remains within the gingival crevice for a sufficient duration to permit dissolution and release of its medicament payload, allowing absorption through the junctional epithelium. However, achieving adequate retention within the gingival crevice presents significant challenges inherent to the anatomical configuration of the gingival crevice. For example, the anatomical configuration of the gingival crevice 550, particularly its V-shape which narrows towards the apical region adjacent to the junctional epithelium 557, naturally predisposes any deposited object, such as a medicament seed, to displacement forces directed upwards toward the broader opening of the gingival crevice. This tendency towards displacement may be further compounded by the dynamic oral environment, including the continuous flow of saliva and gingival crevice fluid, and movements of adjacent tissues, which may contribute to the premature migration of the medicament seed out of the gingival crevice 550 before it has fully dissolved and released its therapeutic payload.

FIG. 1I illustrates a front view of the gingival crevice anatomy of a patient that may be targeted for medicament deposition. In particular, FIG. 1I shows a front view of teeth 555 and 556 along with their respective gingival crevices 550 and 551, illustrating their spatial relationship and the target area for medicament delivery. FIG. 1I illustrates the alignment of the teeth 555 and 556 and the adjacent gingival tissues, showing the positioning of the gingival crevices 550 and 551 where medicament seeds are targeted for deposition. The front view shows the relative placement of the gingival crevices 550 and 551 with respect to one another, providing an overall context of the oral anatomical environment and showing specific areas that may serve as targets for localized medicament delivery in accordance with embodiments of the present disclosure. It is noted that, a medicament seed deposited into one or more of the gingival crevices 550 and 551 may have a natural tendency to be displaced upwards toward the broader, upper region of the gingival crevice. For example, a medicament seed deposited into the gingival crevice 550 may have a natural tendency to be displaced upwards in direction 185 and a medicament seed deposited into the gingival crevice 551 may have a natural tendency to be displaced upwards in direction 186, which may cause the medicament seeds to migrate out of the respective gingival crevice before the medicament may be released.

In embodiments, the medicament seed may be configured for enhanced retention within the gingival crevice. In these embodiments, the medicament seed may operate as a carrier for therapeutic agents, such as micronutrient supplements, medicines, vaccines, etc., which may be intended for controlled release directly into the gingival crevice. By depositing the medicament seed into the targeted gingival crevice, the medicament seed may release its medicament payload gradually, ensuring that the therapeutic agent is efficiently absorbed through the permeable tissues of the crevicular and junctional epithelia.

In some embodiments, the medicament seed may be configured to provide a sustained and controllable release of the medicament. This controlled release mechanism may facilitate optimal bioavailability, as it may allow for the medicament to be absorbed over a predetermined period, reducing the need for frequent re-administration and enhancing the overall therapeutic efficacy. By focusing on the gingival crevice as the site of delivery (and in some embodiments in particular on the junctional epithelium), the disclosed system bypasses the challenges associated with other routes, such as degradation in the gastrointestinal tract, ensuring that a higher concentration of the active substance in the medicament reaches systemic circulation.

Nonetheless, retaining the medicament seed within the gingival crevice poses significant challenges. As discussed elsewhere herein, the inherent V-shaped anatomy of the gingival crevice and its dynamic, moist environment may contribute to the preferential displacement of the medicament seed from its intended site of deposition. To address these challenges, the medicament seed of embodiments may be configured for enhanced retention to ensure that the medicament seed remains securely in place within the gingival crevice until the medicament is fully released into the gingival crevice. The following description includes description of various medicament seed configurations that address these challenges.

The medicament seed may be configured to carry and deliver medicaments to the gingival crevice of the patient. For example, the medicament seed may be configured to, upon deposition into the gingival crevice of the patient, release medicament in a controlled manner, ensuring that the medicament is absorbed into the patient’s system through the gingival crevice efficiently and effectively.

In embodiments, the medicament seed may have a semi-elastic or solid structure. This semi-elastic or solid structure may provide stability to the medicament seed during handling and insertion, while allowing for controlled dissolution once placed in the gingival crevice environment. In embodiments, a semi-elastic nature of the medicament seed may allow for some deformation during insertion, which may facilitate easier placement into the gingival crevice. In some embodiments, a solid structure may provide a more defined shape and controlled release profile.

In embodiments, the medicament seed may be composed of various materials, including polymers. The polymers may be selected based on their biocompatibility, dissolution properties, ability to carry and release the desired medicament, etc. In some embodiments, the medicament seed may be made from biodegradable polymers that break down naturally in the body over time. Examples of such polymers may include polylactic acid (PLA), polyglycolic acid (PGA), or copolymers of these materials.

In some embodiments, the medicament seed may be composed of hydrophilic polymers that absorb moisture from the gingival crevice environment, which may lead to swelling and eventual dissolution of the medicament seed. This particular property may allow for a gradual release of the medicament as the polymer matrix breaks down. In other embodiments, the medicament seed may be made from hydrophobic polymers that dissolve more slowly, which may provide a longer-lasting release of the medicament.

In embodiments, the size of the medicament seed may be configured to facilitate effective delivery and placement within the gingival crevice, while avoiding discomfort to the patient. In particular embodiments, the medicament seed may have a size ranging from 50 to 2000 microns (e.g., in diameter, length, width, and/or height). In some particular embodiments, such as where the medicament seed has an elongated shape, such as oblong, rectangular, or cylindrical configurations, the length of the medicament seed may range from 1 to 20 mm. These dimensions may enable optimal positioning of the medicament seed within the gingival crevice while providing sufficient volume for carrying an effective amount of medicament. The specific size and shape of the medicament seed may be selected based on factors such as the target treatment area, the type and amount of medicament to be delivered, the individual patient’s gingival crevice anatomy.

In some embodiments, the medicament seed may be multi-modal. For example, the medicament seed may be composed or made up of several smaller sub-seeds. In embodiments, each of the sub-seeds may be configured to carry different types or amounts of medicament. In some embodiments, the sub-seeds within the multi-modal medicament seed may vary in size, shape, and/or composition. In some embodiments, each sub-seed may be configured to dissolve at a different rate, allowing for a multi-stage release of medicaments over time. For example, one sub-seed may be formulated to dissolve rapidly, providing an initial burst of medication, while others may be designed for slower, sustained release.

In some embodiments, the multi-modal medicament seed may include sub-seeds with different physical properties. For example, some sub-seeds may be hydrophilic, readily absorbing moisture and dissolving quickly, while others may be hydrophobic, dissolving more slowly. In some embodiments, the sub-seeds may be arranged in layers, with each layer designed to dissolve sequentially. In other embodiments, the sub-seeds may be distributed randomly throughout the medicament seed, which may provide a more uniform release of different medicaments over time. It is noted that a medicament seed with a multi-modal structure may enable allow the incorporation of incompatible medicaments within a single medicament seed. By encapsulating different medicaments in separate sub-seeds, it may be possible to deliver combinations of medicaments that may otherwise be chemically incompatible if mixed directly.

In some embodiments, the multi-modal medicament seed may include sub-seeds with different functions. For example, some sub-seeds may be configured to enhance adhesion to the gingival crevice, while others may be formulated to modify the local environment to improve the efficacy of the delivered medicaments.

In some embodiments, the medicament seed may be coated with a palatable substance to enhance patient comfort and acceptance. This coating may mask any unpleasant taste or texture of the medicament, potentially improving patient compliance with the treatment. In embodiments the palatable coating may be designed to dissolve quickly in the oral environment, allowing the medicament seed to begin its therapeutic action promptly after deposition in the gingival crevice.

In embodiments, the medicament seed may be configured to contain, include, encapsulate, or otherwise carry the medicaments to be delivered to the patient. In embodiments, the medicament seed may be configured to begin its function of medicament delivery upon deposition into the gingival crevice. This release of the medicament into the gingival crevice by the dissolution of the medicament seed may range from immediate release to a sustained release spanning seconds, minutes, hours, or even days.

In embodiments, the medicament seed may be configured to contain medicament in encapsulated polymer (e.g., entrapped in nanoparticle spheres), which include nanoparticle spheres encapsulating or entrapping medicament therein. In some embodiments, free form medicament (e.g., unencapsulated within nanoparticle spheres) may be concurrently contained in the solvent containing nanoparticles.

In some embodiments, the medicament seed may be loaded with micronutrients that include, but are not limited to, vitamins such as retinoid and carotene (Vitamin A), B-complex vitamins like thiamin (Vitamin B1), riboflavin (Vitamin B2), niacin (Vitamin B3), pantothenic acid (Vitamin B5), pyridoxine (Vitamin B6), cobalamin (Vitamin B12), biotin, and folic acid (Vitamin B9), ascorbic acid (Vitamin C), vitamins D (including calciferol, calcifediol, cholecalciferol, and ergocalciferol) and E (alpha-tocopherol), and Vitamin K, including phylloquinone and menadione. The micronutrients may include essential minerals such as calcium, chloride, chromium, copper, fluoride, iodine, iron, magnesium, manganese, molybdenum, phosphorus, potassium, selenium, sodium, sulfur, and zinc, and/or antibiotics such as amikacin, betamethasone, clindamycin, clotrimazole, gentamicin, kanamycin, minocycline, oxytetracycline, penicillin, and tetracycline.

In some embodiments, the medicament seed may include anti-inflammatory agents, antimicrobials, antifungals, and antibodies to combat various pathogens. Steroids can be included for their potent anti-inflammatory effects, while antiparasitic, anti-amoebic, anti-helminthic, anti-protozoal, antinematode, anti-cestode, and anti-trematode agents can be used to treat specific parasitic infections.

In some embodiments, the medicament seed may include vaccine/s to combat various pathogens. In this manner, the medicament seed of embodiments may be used for immunomodulation to combat future infections (such as, without limitation, influenza, human papilloma virus, coronavirus, etc.) by enabling the delivery of controlled doses of the vaccine/s directly to the gingival crevice specifically targeting the junctional epithelium.

In embodiments, the medicament seed may include allergy-inducing antigens, specific allergy-inducing molecules, or any combination thereof, to act as allergenic tolerance therapies, or to act as vaccines. In this manner, the medicament seed of embodiments may be used to treat and modulate tolerance to food allergies, such as peanut or shellfish allergies, as well as vaccinate individuals, by enabling the delivery of controlled doses of the allergens directly to the gingival crevice.

In embodiments, the medicament seed may be dissolvable. In this manner, the medicament seed may be configured to degrade to molecular form over a period of time after being deposited into the gingival crevice. In some embodiments, the dissolution of the medicament seed may allow for controlled release of the medicament into the surrounding tissue. The rate of dissolution of the medicament seed may vary depending on factors such as the composition and structure of the medicament seed, the environmental conditions in the gingival crevice, the specific medicament being delivered, etc. In some embodiments, the medicament seed may be designed to dissolve completely, leaving no residue in the gingival crevice after the medicament has been fully released.

In some other embodiments, the medicament seed may be non-dissolvable and may remain within the gingival crevice after delivering the medicament. In these embodiments, the medicament seed may be configured to release the medicament over time while maintaining structural integrity. After the medicament has been fully delivered, the non-dissolvable medicament seed may need to be removed from the gingival crevice. This removal process may include a separate procedure or the use of specialized tools to extract the medicament seed from the gingival crevice.

In embodiments, the medicament seed may be composed, in whole or in part, of a dissolvable polymer that is selected for its biocompatibility and dissolution characteristics. A variety of polymers can be utilized for this purpose, including but not limited to hydroxy acids, lactic acid polymers, glycolic acid polymers, poly (hydroxyl acids), poly(lactic acid) (PLA), poly(glycolic acid) (PGA), poly(lactic-co-glycolic acid) (PLGA), and copolymers with PEG, polyanhydrides, poly(ortho) esters, polyesters, polyurethanes, poly(butyric acid), poly(valeric acid), poly(caprolactone), poly (hydroxyalkanoates), poly(lactide-co-caprolactone), blends and copolymers thereof, natural polymers, proteins, albumin, collagen, gelatin, prolamines, zein, polysaccharides, alginate, cellulose derivatives and polyhydroxyalkanoates, polyhydroxy butyrate blends, and copolymers thereof.

In additional or alternative embodiments, natural polymers such as proteins, albumin, collagen, gelatin, prolamines, zein, and polysaccharides like alginate, as well as cellulose derivatives and polyhydroxyalkanoates, polyhydroxy butyrate blends, and copolymers thereof and other biologics, can be employed to form the medicament seed.

In embodiments, the polymers used in the construction of the medicament seed may be processed using solvents such as dichloromethane, ethyl acetate, acetone, or a combination thereof, to achieve the desired form and dissolution rate. As such, the medicament seed’s configuration incorporates a dissolvable polymer matrix that responds to the environment of the gingival crevice providing functionality of the medicament seed to deliver medicaments over a controllable period of time. This configuration ensures that the medicament is released at a predetermined rate, providing a consistent and targeted therapeutic effect directly within the gingival crevice.

In embodiments, the rate of dissolution of the medicament seed may be configured to specific therapeutic requirements by adjusting the composition and properties of the medicament seed. For example, the rate of dissolution of the medicament seed may be configured by varying the type of polymer used for constructing the medicament seed, the concentration of the polymer (e.g., the density) of the medicament seed, the molecular weight of the polymer used for the medicament seed, the chain functional groups contents in the medicament seed, etc. In embodiments, the rate of dissolution may be increased or decreased by varying the above features. In some embodiments, the medicament seed may be configured with a multi-layered structure, where each layer has different dissolution properties, which may allow for phasic release of the medicament.

In embodiments, the rate of dissolution of the medicament seed may be configured based on the characteristics of the medicament seed, which may include the composition, structure, and/or surface energy of the polymer used in its construction. For example, for a slow and steady release of the medicament, the polymer may be selected or treated to dissolve at a slower rate, which may slow down the rate of dissolution in the moist environment of the gingival crevice. Conversely, for a more rapid release, the polymer may be treated or composed to dissolve at a faster rate subsequently increasing the rate of release of the medicament.

In embodiments, the medicament seed may be configured to carry the medicament using surface coating, in which at least a portion of the surface of the medicament seed may be coated with the medicament. In some embodiments, the medicament may be uniformly coated across the at least a portion of the surface of the medicament seed. In some embodiments, multiple coatings may be deposited over the surface of the medicament seed.

In embodiments, prior to coating with the medicament, a precoat layer may be applied to the medicament seed to modify the surface energy of the medicament seed, which may influence the dissolution rate and the subsequent release of the medicament. The precoat layer may also contain medicaments, which could be in free form, entrapped or encapsulated in particles (e.g., nanoparticles), encapsulated, or a combination thereof.

In embodiments, the medicaments with which the medicament seed may be coated may include medicaments in free form, entrapped or encapsulated in particles (e.g., nano-particles, encapsulated, or a combination thereof. In embodiments, the encapsulant for the medicament is selected from one or more polymers of the group consisting of poly(hydroxy acids), poly(lactic acid), Poly(glycolic acid), poly(lactic acid-co-glycolic acid), poly(lactic-co-glycolic acid), derivatives of poly(lactic-co-glycolic acid), PEGylated poly(lactic-co-glycolic acid), poly(lactide), poly(glycolide), poly(lactide-co-glycolide), poly(anhydrides), PEGylated poly(anhydrides), poly(ortho esters), derivatives of poly(ortho esters), PEGylated poly(ortho esters), poly(caprolactones), derivatives of poly(caprolactone), PEGylated poly(caprolactones), polyamines, spermine, spermidine, polylysine, and derivatives thereof, PEGylated polylysine, polyamides, polycarbonates, poly(propylene fumarates), polyamides, polyphosphazenes, polyamino acids, polyethers, polyacetals, polylactides, polyhydroxyalkanoates, polyglycolides, polyketals, polyesteramides, poly(dioxanones), polyhydroxybutyrates, polyhydroxyvalyrates, polycarbonates, polyorthocarbonates, poly(vinyl pyrrolidone), polycyanoacrylates, polyalkylene oxalates, polyalkylene succinates, poly(malic acid), poly(methyl vinyl ether), poly(ethylene imine), poly(acrylic acid), poly (maleic anhydride), poly(ethylene imine), derivatives of poly(ethylene imine), PEGylated poly(ethylene imine), poly (acrylic acid), derivatives of poly(acrylic acid), PEGylated poly(acrylic acid), poly(urethane), PEGylated polyurethane), derivatives of poly(urethane), poly (lactide), poly (glycolide), poly (hydroxyacids), polyesters, poly(arylates), polyalkylenes, polyethylene, polypropylene, polyalkylene glycols, poly(ethyleneglycol), polyalkylene oxides such as poly(ethylene oxide), polyalkylene terepthalates, poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinylacetate), polystyrene, polyurethanes and co-polymers thereof derivatized celluloses, alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutylmethyl cellulose, cellulose acetate, cellulose propionate, cellulose acetate butyrate, cellulose acetate phthalate, carboxylethyl cellulose, cellulose triacetate, cellulose sulfate sodium salt, polymers of acrylic acid, methacrylic acid or copolymers or derivatives thereof, methacrylic acid esters, poly(methyl methacrylate), poly(ethyl methacrylate), poly(butylmethacrylate), polyisobutyl methacrylate), poly(hexylmethacrylate), poly(isodecyl methacrylate), poly(laurylmethacrylate), poly(phenyl methacrylate), poly(methylacrylate), poly(isopropyl acrylate), poly(isobutyl acrylate), and poly(octadecyl acrylate), poly(butyric acid), poly(valeric acid) , and poly(lactide-co caprolactone ) and/or derivatives thereof, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methylmethacrylate) copolymer, polyacrylamide, aminoalkyl methacrylate copolymer, glycidyl methacrylate copolymers, polycyanoacrylates, and/or combinations thereof, natural polymers, proteins (such as albumin, collagen, gelatin), prolamines (for example, zein), polysaccharides (such as alginate), cellulose derivatives (such as hydroxypropyl cellulose, sodium carboxymethyl cellulose, ethyl cellulose and cellulose acetate), polyhydroxyalkanoates (for example, poly(hydroxybutyrate), and/or combinations thereof.

In embodiments, the medicament seed may be configured to carry the medicament using a polymer/medicament mixture, which may include integrating the medicament directly into the polymer matrix to form a polymer/medicament mixture. The polymer/medicament mixture may be used to form or make the medicament seed. In this manner, the medicament is not merely coated on the surface of the medicament seed but is distributed throughout the polymer material, making it an intrinsic part of the medicament seed’s composition.

In embodiments, the polymer used for the polymer/medicament mixture may be a biocompatible, biodegradable polymer that is well-tolerated within the oral environment. The selection of the polymer may be based on its dissolution properties, compatibility with the medicament, and the desired release profile. Polymers such as poly(lactic acid), poly(glycolic acid), and their copolymers, including poly(lactic-co-glycolic acid), may be used due to their ability to degrade into non-toxic byproducts.

In embodiments, the medicament seed may be configured to carry the medicament using an internal well. For example, in embodiments, an internal well may be formed within the interior of the medicament seed. The internal well may be configured to encapsulate or trap the medicament within, which may be in liquid form or another suitable state for controlled release. The medicament seed may be configured to degrade or dissolve at a predetermined rate once placed within the moist environment of the gingival crevice. As the medicament seed begins to dissolve, it eventually breaches the internal well, allowing the encapsulated medicament to escape and be released into the gingival crevice. This release mechanism is activated by the dissolution of the medicament seed, which may be calibrated to occur over a controllable period.

In embodiments, the medicament seed 150 may be configured to carry the medicament using internal thread-like structures (monofilament) or mesh. In some embodiments, each thread of the internal thread-like structure may carry a different medicament portion and/or a different type of medicament. In some embodiments, these thread-like structures may be incorporated into a housing configured to dissolve. In embodiments, the housing and the internal thread-like structures may form a medicament seed.

In some embodiments, the internal well of the medicament seed may be configured to hold multiple types of medicaments, allowing for a combination therapy approach. For example, in some embodiments, the internal well may include multiple sub-wells, each sub-well configured to encapsulate and/or trap a different type and/or dosage of medicament. In some embodiments, each of the sub-wells may be configured to breach at different dissolution times of the medicament seed, which may allow for a multi-stage delivery of the medicament dosages. In some embodiments, the multiple sub-wells may be laid out side-by-side, or may be laid out one within the other (e.g., a first sub-well may be encapsulated by a second sub-well, etc.).

In embodiments, the medicament seed may be configured to carry the medicament using a multi-stage delivery configuration. The multi-stage delivery configuration may represent a combination of the features of the polymer/medicament mixture configuration and the internal well configuration. In the multi-stage delivery configuration, the medicament seed may be composed of a polymer/medicament mixture, and additionally, an internal well may be incorporated to encapsulate additional medicament. This configuration allows for a sequential release of medicaments. For example, in a first stage of the multi-stage delivery configuration of the medicament seed, as the medicament seed begins to dissolve within the gingival crevice, the medicament embedded within the polymer/medicament mixture may be gradually released. A second stage of the multi-stage delivery configuration may be initiated once the dissolution process reaches the internal well of the medicament seed, breaching its integrity and allowing the encapsulated medicament to be released into the gingival crevice. This second stage can be configured to occur after a predetermined time interval following the initial release, providing a subsequent boost or a different therapeutic action as dictated by the treatment plan. The encapsulated medicament within the internal well may include the same type of medicament as in the polymer/medicament mixture for a sustained dosage or may include a different type of medicament to address additional aspects of the patient’s condition.

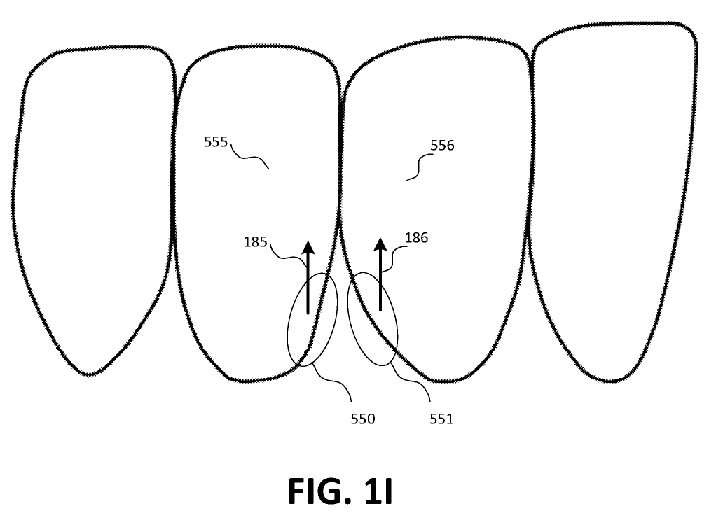

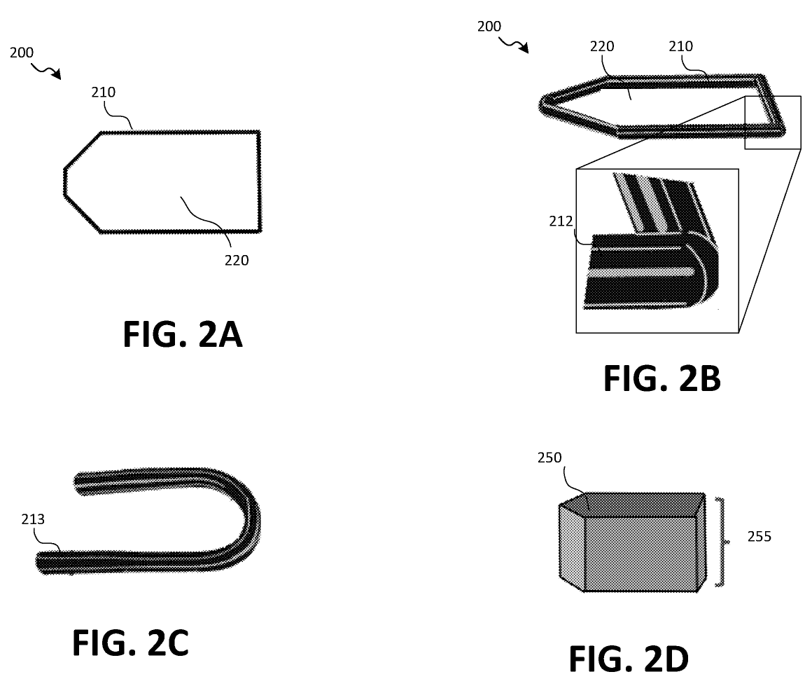

It is noted that any of the above described features and/or characteristics of the medicament seed may be used for any of the configurations of the medicament seeds or medicaments described with respect to FIGS. 2A-17.

Various embodiments of the present disclosure are directed to devices, systems, and techniques for loading medicament onto a medicament carrier. In embodiments, the medicament may be loaded onto a carrier frame of the medicament carrier using a transphasic molding technique. Transphasic molding of the carrier may include configuring the medicament such that the medicament transitions through one or more physical phases during the loading of the medicament onto the carrier frame and/or during subsequent delivery of the medicament into the gingival crevice.

In embodiments, the medicament may initially be provided as part of a medicament solution. The medicament solution may include the medicament, a solvent, and/or one or more excipients. The excipients may include, for example, one or more plasticizers, hygroscopic agents, emulsifiers, viscosity enhancers, surfactants, stabilizers, binders, and/or other components. In embodiments, a viscosity enhancer may be configured to control the viscosity of the medicament solution so as to enhance surface tension behavior and control flow characteristics. In embodiments, a surfactant may be configured to modify the surface energy of the medicament solution so as to enable the medicament solution to attain and maintain the shape of a medicament well defined by the carrier frame. In embodiments, a plasticizer may be configured to facilitate flexibility, binding, and structural cohesion of the medicament after solvent dissipation.

In embodiments, the medicament solution may be applied onto the carrier frame using one or more techniques. For example, the medicament solution may be applied using drop coating, dip coating, spray deposition, controlled dispensing, and/or other deposition techniques. In additional or alternative embodiments, the medicament may be associated with a dissolvable membrane configured to wrap around or otherwise interface with the carrier frame to hold the medicament in place within the medicament well. The dissolvable membrane may be configured to dissolve upon exposure to moisture within the gingival crevice, releasing the medicament.

In embodiments, once the medicament solution is applied onto the carrier frame, the medicament solution may initially be in a flowable or semi-flowable phase. The medicament solution may fill the medicament well formed by the geometry of the carrier frame. During this initial phase, surface tension of the medicament solution may cause the medicament solution to “wet” the medicament well. In embodiments, the wetting behavior may allow the medicament solution to spread across and fill the enclosed region defined by the carrier frame. The surfactant, viscosity enhancer, plasticizer, and other excipients within the medicament solution may facilitate this wetting behavior and may enable the medicament solution to conform to the geometry of the medicament well.

As the solvent within the medicament solution dissipates, evaporates, diffuses, or otherwise transitions away from the medicament solution, the medicament may remain within the medicament well. In embodiments, during this transphasic transition from a flowable phase to a more solid or semi-solid phase, the plasticizer and other excipients may operate to maintain structural integrity of the medicament within the medicament well. The resulting molded medicament may retain the geometry defined by the medicament well and may remain secured within the carrier frame.

It is noted that the surface tension characteristics of the medicament solution may be critical for proper loading. In embodiments, if the surface tension of the medicament solution is too high, the medicament solution may not adequately wet the medicament well. For example, the medicament solution may bead or retract rather than fill the medicament well. In such embodiments, the surfactant within the medicament solution may reduce interfacial tension and may allow the medicament solution to attain and maintain the shape of the medicament well. In embodiments, the plasticizer and other binding components may further facilitate cohesion of the medicament within the medicament well such that substantially no component of the medicament solution is lost during loading or solvent dissipation.

FIGS. 2A–2C illustrate examples of a medicament carrier 200 configured to carry medicament for deposition into a gingival crevice of a patient for absorption through the junctional epithelium in accordance with embodiments of the present disclosure. In embodiments, the medicament carrier 200 may represent a linear framework configuration that is configured to define a medicament well 220 using a carrier frame 210. In embodiments, the medicament well 220 defined by the carrier frame 210 may be configured to receive medicament (e.g., a medicament seed) using a transphasic loading technique as described herein.

In embodiments, the medicament (e.g., the medicament or medicament seed) may initially be provided in a medicament solution phase and may be applied onto the carrier frame 210 so that the medicament solution fills the medicament well 220 formed by the geometry of the carrier frame 210. During an initial phase of loading, surface tension of the medicament solution may cause the medicament solution to wet and conform to the medicament well 220. Thereafter, as a solvent within the medicament solution dissipates, the medicament may transition into a more solid or semi-solid phase while remaining retained within the medicament well 220. In this manner, the medicament carrier 200 shown in FIGS. 2A–2C may be configured not only to define the medicament well 220 geometrically, but also to facilitate transphasic molding of the medicament within the medicament well 220 prior to insertion into the gingival crevice

As shown in FIG. 2A, the carrier frame 210 may be formed from a filament that is shaped into a predetermined planar geometry that defines the medicament well 220. In embodiments, the carrier frame 210 may lie substantially within a single plane such that the medicament well 220 is defined by the enclosed area bounded by the filament forming the carrier frame 210. In this linear framework configuration, the medicament well 220 may not include volumetric depth in a three-dimensional sense. Rather, the thickness dimension of the medicament well 220 may correspond substantially to the thickness of the filament forming the carrier frame 210.

In embodiments, because the carrier frame 210 lies within a single plane, the loading of medicament into the medicament well 220 using transphasic molding may rely substantially on surface tension characteristics of the medicament solution during the initial loading phase. For example, when the medicament solution is applied onto the carrier frame 210 (e.g., by drop coating or dip coating), the surface tension of the medicament solution may cause the medicament solution to wet, spread across, and fill the medicament well 220 defined by the planar geometry of the carrier frame 210. The planar configuration may facilitate predictable wetting behavior, as the medicament solution may conform to the perimeter defined by the carrier frame 210 and may be retained across the medicament well 220 due to cohesive forces within the medicament solution and adhesive forces relative to the carrier frame 210.

FIG. 2B further illustrates that the carrier frame 210 may be formed from a filament having a defined thickness 212. The thickness 212 of the filament may define the structural thickness of the carrier frame 210. In embodiments, during the initial phase of transphasic loading, the medicament solution may occupy the medicament well 220 while in a flowable or semi-flowable phase. As solvent within the medicament solution dissipates, evaporates, or otherwise transitions away, the medicament may transition into a more solid or semi-solid phase while remaining retained within the medicament well 220. Because the carrier frame 210 lies within a single plane, the final thickness of the molded medicament may be controlled by the thickness 212 of the filament and the volume of medicament initially retained within the medicament well 220 during the wetting phase.

In embodiments, the overall thickness of the medicament carrier 200 after transphasic molding may be defined by the combined thickness of the carrier frame 210 (e.g., the filament thickness 212) and the thickness of the medicament retained within the medicament well 220 after solvent dissipation. In embodiments, this combined thickness may be configured to be less than 1 mm. In particular embodiments, the combined thickness may be configured to be less than 600 microns. This dimensional control may be facilitated by the planar linear framework architecture of the carrier frame 210, which may allow precise control over filament thickness 212 and predictable formation of the molded medicament within the medicament well 220.

In embodiments, maintaining the overall or combined thickness within this dimensional range may enable the medicament carrier 200 to be inserted into the gingival crevice and advanced to the base of the gingival sulcus or crevice proximate to the junctional epithelium without causing trauma to the gingival tissues. If the combined thickness exceeds this range, the medicament carrier 200 may not be capable of reaching the apical region of the gingival crevice, or may require excessive insertion force that may damage the gingival crevice. In such a case, the medicament would not be positioned proximate to the junctional epithelium, and effective targeting of the junctional epithelium for absorption would not be achieved.

In embodiments, the carrier frame 210 may be composed of various materials selected based on biocompatibility, structural integrity, dissolution characteristics, manufacturability, and/or interaction with the medicament and the gingival crevice environment. In embodiments, the carrier frame 210 may be formed from a monofilament, braided filament, semi-rigid material, or other structural member configured to define the medicament well 220.

In embodiments, where the carrier frame 210 is formed from a monofilament, the monofilament may include a single strand or string-like filament having a thickness of approximately 25 to 600 microns. In embodiments, the carrier frame 210 may alternatively be formed from a braided filament composed of multiple filament strands, each strand having a thickness between approximately 25 to 120 microns. The braided filament configuration may provide increased surface area, enhanced flexibility, and/or increased mechanical strength relative to a monofilament configuration. In embodiments, the braided filament configuration may also provide increased micro-texturing that may facilitate retention of medicament during transphasic loading.

In embodiments, the carrier frame 210 may be formed from a semi-rigid material, which may include a polymer configured to be shaped using a molding process, stamping process, extrusion process, or additive manufacturing process. In embodiments, when the carrier frame 210 is formed from filament, the geometry of the carrier frame 210 may be created by folding, bending, looping, or otherwise shaping the filament into the desired planar configuration that defines the medicament well 220. When formed from a molded or stamped polymer, the carrier frame 210 may be manufactured directly into the desired planar geometry without requiring bending of filament.

In embodiments, the carrier frame 210 may be composed of a biopolymer, biodegradable polymer, synthetic polymer, natural polymer, composite material, plastic material, metal material, or combinations thereof. In embodiments, the carrier frame 210 may be composed of a dissolvable material configured to dissolve within the gingival crevice after deposition. For example, the carrier frame 210 may be formed from a polymer selected for its ability to dissolve, degrade, or bioresorb over a controlled period after placement within the gingival crevice. In such embodiments, the carrier frame 210 may be configured to remain within the gingival crevice and dissolve along with the medicament seed.

In embodiments, the carrier frame 210 may be infused with at least a portion of the medicament to be delivered. For example, the material forming the carrier frame 210 may include medicament dispersed throughout the polymer matrix of the filament or molded structure. In such embodiments, the carrier frame 210 itself may contribute to delivery of medicament in addition to the medicament retained within the medicament well 220.

In embodiments, the carrier frame 210 may include the medicament itself reinforced with mucoadhesive components, strengthening agents, fibers, and/or structural additives to enhance mechanical strength. In embodiments, the carrier frame 210 may be formed partially or entirely from a medicament-containing polymer composition that is configured to maintain structural integrity during insertion and subsequently dissolve within the gingival crevice.

In embodiments, the geometry of the carrier frame 210 may be selected from a wide variety of planar shapes. For example, the carrier frame 210 may be configured in cylindrical, circular, oval, square, rectangular, triangular, polygonal, cubical perimeter projection, and/or other geometric configurations. The planar loop geometry may define the medicament well 220 of varying shape and surface area depending on the intended medicament volume and insertion profile.

FIG. 2C illustrates another embodiment of a carrier frame 213 configured as a linear framework. In this embodiment, the carrier frame 213 may include a different planar loop geometry, such as a generally U-shaped configuration. Similar to the carrier frame 210 described with respect to FIGS. 2A and 2B, the carrier frame 213 may be formed from a filament having a defined thickness and may lie substantially within a single plane. The planar geometry of the carrier frame 13 may define a region configured to operate as a medicament well when medicament is loaded thereon using transphasic molding.

In embodiments, the carrier frame 213 may similarly facilitate surface tension-based wetting of a medicament solution during an initial loading phase, followed by phase transition of the medicament as solvent dissipates. The combined thickness of the carrier frame 213 and the molded medicament retained thereon may likewise be configured to be less than 1 mm, and preferably less than 600 microns, to enable insertion to the base of the gingival sulcus proximate to the junctional epithelium. In embodiments, the specific planar geometry of the carrier frame 213 may be selected based on loading characteristics, manufacturing considerations, insertion dynamics, or retention behavior within the gingival crevice, while maintaining the dimensional constraints necessary to reach and target the junctional epithelium.

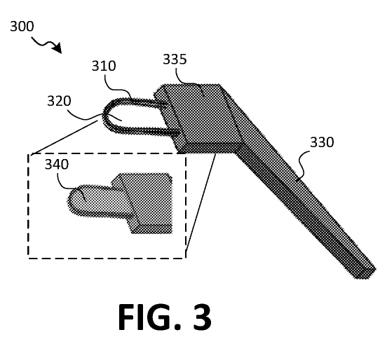

FIG. 3 illustrates an example system 300 representing an implementation of a linear framework configuration for delivering medicament into a gingival crevice of a patient for absorption through the junctional epithelium in accordance with embodiments of the present disclosure. In embodiments, the system 300 may include a handle 330, a head 335 coupled to the handle 330, and a carrier frame 310 coupled to the head 335. The carrier frame 310 may define a medicament well 320 configured to receive and retain medicament using transphasic loading as described herein.

In embodiments, the handle 330 may be configured to operate as a user interface for manipulating, positioning, and/or inserting the carrier frame 310 into a gingival crevice. The handle 330 may be configured to provide structural rigidity and directional stability during insertion. The head 335 may be coupled to a distal end of the handle 330 and may be configured to support the carrier frame 310. In embodiments, the head 335 may be configured to position the carrier frame 310 at a desired orientation relative to the handle 330 so that the carrier frame 310 may be advanced into the gingival crevice to reach the base of the gingival crevice in a controlled manner.

In embodiments, the carrier frame 310 may be configured as a linear framework lying substantially within a single plane, similar to the linear framework configurations described with respect to FIGS. 2A–2C. The carrier frame 310 may be formed from a filament, strand, and/or semi-rigid polymer configured to define the medicament well 320. In embodiments, the medicament well 320 may correspond to the enclosed region bounded by the geometry of the carrier frame 310.

In embodiments, the medicament 340 may be loaded onto the carrier frame 310 (as shown in the insert in FIG. 3). In embodiments, the medicament 340 may initially be included in a medicament solution comprising medicament, solvent, and/or one or more excipients such as plasticizers, viscosity enhancers, surfactants, hygroscopic agents, or emulsifiers. The medicament solution may be applied to the carrier frame 310 by drop coating, dip coating, controlled dispensing, and/or other loading techniques. In embodiments, during an initial loading phase, the medicament solution may wet and fill the medicament well 320 due to surface tension relative to the carrier frame 310.

In embodiments, as solvent within the medicament solution dissipates, evaporates, or otherwise transitions away, the medicament 340 may undergo a phase transition from a flowable or semi-flowable state to a more solid or semi-solid state. During this transphasic transition, the medicament 340 may remain retained within the medicament well 320 defined by the carrier frame 310. Plasticizers and other excipients within the medicament formulation may facilitate maintaining the medicament 340 within the medicament well 320 during solvent dissipation and subsequent structural stabilization.

In embodiments, after completion of the transphasic loading process, the medicament 340 retained within the medicament well 320 of the carrier frame 310 may form a medicament seed. The medicament seed may be configured to remain structurally integrated with the carrier frame 310 during handling and insertion. The planar linear framework architecture of the carrier frame 310 may allow dimensional control over the combined thickness of the carrier frame 310 and the medicament 340, such that the overall thickness may be less than 1 mm, and preferably less than 600 microns, as described herein. This dimensional configuration may enable the carrier frame 310 carrying the medicament 340 to be advanced to the base of the gingival sulcus proximate to the junctional epithelium without causing trauma to the gingival tissue.

In embodiments, once the medicament 340 has been transphasically molded within the medicament well 320 and stabilized as a medicament seed on the carrier frame 310, the system 300 may be configured to position the carrier frame 310 within the gingival crevice. The handle 330 may be manipulated to insert the carrier frame 310 into the gingival crevice until the carrier frame 310 reaches the base of the gingival crevice proximate to the junctional epithelium. In embodiments, after insertion, the carrier frame 310 may be detached from the head 335 (e.g., via a release interface described elsewhere herein) such that the carrier frame 310 and the medicament 340 remain within the gingival crevice for absorption through the junctional epithelium.

In some embodiments, the carrier frame 310 may not detach from the head 335, but may instead remain attached. In these embodiments, however, after insertion, the medicament 340 may dislodge from the carrier frame 310 and may be delivered into the base of the gingival crevice for absorption through the junctional epithelium.

In embodiments, transphasic molding of the medicament carrier may include configuring the carrier frame with a plurality of compartments defined within the geometry of the medicament well. Rather than forming a single continuous medicament well, the carrier frame may be configured to include one or more partitioners that subdivide the medicament well into a plurality of compartments. Each compartment may be configured to receive medicament during transphasic loading and may form part of the medicament seed that is ultimately deposited into the gingival crevice.

In embodiments, the compartments defined within the carrier frame may be configured to release the medicament retained therewithin at the same stage or at different stages. For example, during transphasic molding, a first compartment may be loaded with a medicament composition configured for relatively rapid dissolution upon deposition within the gingival crevice, while a second compartment may be loaded with a medicament composition configured for slower dissolution. In this manner, the carrier frame may be configured to support multi-phasic release profiles within a single medicament seed. In embodiments, the compartments may contain identical medicament compositions, different medicament compositions, different concentrations of the same medicament, or combinations thereof.

In embodiments, each compartment may be configured to receive different medicament compositions. For example, one compartment may be configured to retain a hydrophilic medicament composition while another compartment may be configured to retain a hydrophobic medicament composition. In embodiments, adjacent compartments may be configured to retain substances that react upon contact after solvent dissipation. For example, one compartment may retain a base component and another compartment may retain a catalyst component such that when solvent dissipates and the components contact one another within the gingival crevice, a gel or other structured composition may form. In embodiments, this configuration may enable in situ phase transformation after deposition within the gingival crevice.

In embodiments, different compartments of the carrier arm may carry different liquids. In embodiments, the medicament loaded onto the carrier arm may be added to one of the liquids, and the substance can then get trapped in a formed gel/solid. Examples include (i) substances including but not limited to butyl and octyl cyanoacrylate blends that will rapidly polymerize and solidify in situ when in contact with moisture, (ii) hydrogel forming substances and polymers like polyvinyl alcohol and polyethylene glycol that can be chemically modified to gel in response to moisture, (iii) thermoplastic polymers like polyvinyl pyrrolidone and other thermos-reversible gelation agents that can be formulated as liquids and will undergo phase transition to in response to moisture or temperature, (iv) polysaccharide formulations, such as chitosan, that will form gels when interacting with ions such as calcium found in saliva.

In embodiments, configuring the compartments within the carrier frame may include shaping the carrier frame into particular geometries that define internal partition boundaries. For example, the carrier frame may include one or more internal structural members extending across the medicament well to divide the well into separate regions. These internal structural members may operate as partitioners that define the compartments. The partitioners may be formed integrally with the carrier frame during molding, stamping, or extrusion, or may be formed by additional filament segments connected across the carrier frame when formed from monofilament or braided filament.

In embodiments, when using monofilament to form the carrier frame, the geometry of the compartments may be based on the rigidity and diameter of the monofilament. For example, the degree to which a monofilament may be bent or shaped into complex compartmental geometries may depend on its stiffness and diameter. In embodiments, monofilament rigidity may limit certain geometric configurations, resulting in fewer shape options relative to more flexible materials. However, monofilament configurations may provide predictable dimensional stability and consistent thickness control, which may be advantageous for maintaining the combined thickness constraints necessary to reach the base of the gingival sulcus proximate to the junctional epithelium.

In embodiments, arm frames formed from monofilament or braided filament may include some degree of three-dimensional depth depending on the diameter of the filament used. For example, a monofilament having a diameter of up to approximately 300 microns may provide structural depth to the carrier frame. This structural depth may define the thickness of each compartment and may influence the volumetric capacity of medicament retained within each compartment during transphasic loading.

In embodiments, the different compartments defined within the carrier frame may be filled with different fillings and materials, or all compartments may be filled with the same material. For example, one compartment may be filled with a hydrophilic medicament composition while another compartment may be filled with a hydrophobic medicament composition. In embodiments, one compartment may include a base component and another compartment may include a catalyst component, such that upon solvent dissipation or upon exposure to the gingival crevice environment, the components may interact to form a gel or other structural configuration. In embodiments, the compartments may contain pure drug, pure drug combined with excipients, or drug encapsulated within nanoparticles dispersed within a polymer matrix.

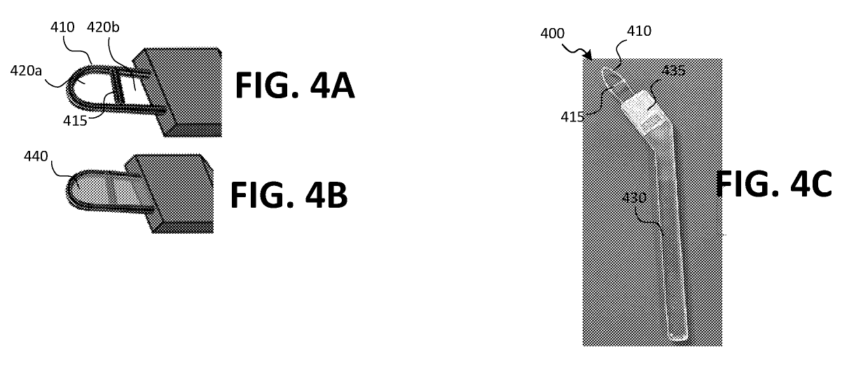

FIGS. 4A–4C illustrate examples of a two-compartment linear framework configuration implemented using a carrier arm 410 in accordance with embodiments of the present disclosure. In embodiments, the carrier arm 410 may represent a planar linear framework architecture similar to the carrier frame 210 described with respect to FIGS. 2A–2C, but further configured with a partitioner 415 that subdivides the medicament well into multiple compartments.

As shown in FIG. 4A, the carrier arm 410 may be configured with a two-compartment geometry including a first compartment 420a and a second compartment 420b. The first compartment 420a and the second compartment 420b may be separated by the partitioner 415. In embodiments, the partitioner 415 may be formed integrally with the carrier arm 410 when manufactured. In embodiments, where the carrier arm 410 is formed from monofilament or braided filament, the partitioner 415 may be formed by an additional cross-member segment extending across the planar geometry of the carrier arm 410.

In embodiments, the partitioner 415 may provide structural reinforcement to the carrier arm 410. For example, without the partitioner 415, the carrier arm 410 defining the medicament well may be susceptible to deformation during handling, loading, insertion, or deployment into the gingival crevice. Such deformation may cause the boundaries of the compartments to become undefined, disrupting transphasic loading behavior or altering the geometry of the medicament seed after solvent dissipation. In embodiments, the partitioner 415 may maintain the structural integrity of the carrier arm 410 and preserve the geometric definition of the first compartment 420a and the second compartment 420b during loading and insertion.

FIG. 4B illustrates the carrier arm 410 after transphasic loading. In this configuration, medicament 440 may be retained within the compartments defined by the carrier arm 410 and the partitioner 415. The medicament 440 may represent the molded medicament seed formed after solvent dissipation from the medicament solution applied during loading. In some embodiments, the partitioner 415 may ensure that the medicament 440 remains geometrically separated between the first compartment 420a and the second compartment 420b.

In embodiments, the combined thickness of the carrier arm 410 and the medicament 440 retained within the compartments may be configured to be less than 1 mm, and preferably less than 600 microns.

FIG. 4C illustrates an example system 400 implementing the two-compartment carrier arm 410 configuration. In embodiments, the system 400 may include a handle 430 and a head 435 configured to support the carrier arm 410. The handle 430 may be configured to facilitate positioning and insertion of the carrier arm 410 into the gingival crevice as described herein. The head 435 may be configured to couple the carrier arm 410 to the handle 430 in a manner that maintains proper orientation during insertion.

In embodiments, after transphasic loading of medicament 440 into the compartments of the carrier arm 410, the system 400 may be used to advance the carrier arm 410 into the gingival crevice until the carrier arm 410 reaches the base of the gingival sulcus proximate to the junctional epithelium. The structural reinforcement provided by the partitioner 415 may prevent deformation of the carrier arm 410 during insertion, preserving the geometric definition of the compartments and ensuring consistent positioning of the medicament 440 at the targeted anatomical location.

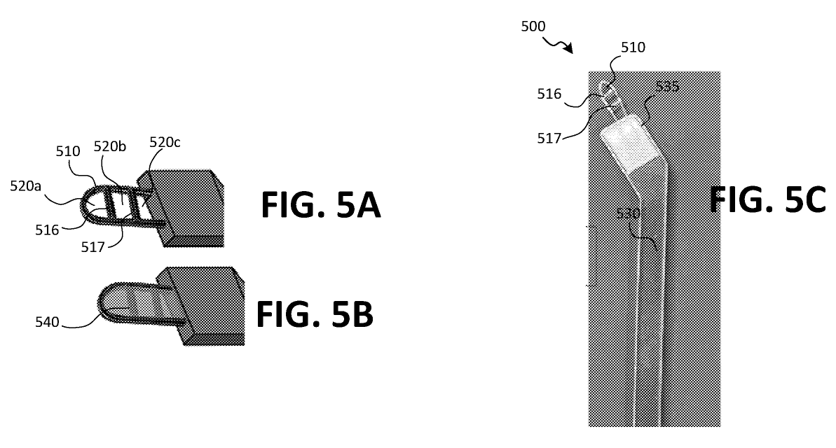

FIGS. 5A–5C illustrate an example of a three-compartment linear framework configuration implemented using a carrier arm 510 in accordance with embodiments of the present disclosure. In embodiments, the carrier arm 510 may be configured with additional partitioners to define three distinct compartments within the medicament well.

As shown in FIG. 5A, the carrier arm 510 may be configured with a first compartment 520a, a second compartment 520b, and a third compartment 520c. The first compartment 520a, the second compartment 520b, and the third compartment 520c may be separated by a first partitioner 516 and a second partitioner 517. In embodiments, the first partitioner 516 and the second partitioner 517 may be formed integrally with the carrier arm 510 when manufactured. In embodiments, when the carrier arm 510 is formed from monofilament or braided filament, the first partitioner 516 and the second partitioner 517 may be formed by additional cross-member segments extending across the planar geometry of the carrier arm 510.

In embodiments, the first partitioner 516 and the second partitioner 517 may provide structural reinforcement to the carrier arm 510. For example, dividing the medicament well into three compartments may reduce unsupported span length across the planar framework, increasing rigidity and resistance to deformation during handling, loading, insertion, and deployment into the gingival crevice. In embodiments, without the first partitioner 516 and the second partitioner 517, the carrier arm 510 may deform during use, which may cause the boundaries of the compartments to become undefined. Such deformation may alter transphasic loading behavior and may disrupt consistent medicament placement within the gingival crevice.

FIG. 5B illustrates the carrier arm 510 after transphasic loading, in which medicament 540 may be retained within the first compartment 520a, the second compartment 520b, and the third compartment 520c. The medicament 540 may represent the molded medicament seed formed after solvent dissipation. In some embodiments, the first partitioner 516 and the second partitioner 517 may maintain geometric separation between the compartments during and after loading.

In embodiments, the combined thickness of the carrier arm 510 and the medicament 540 retained within the compartments may be configured to be less than 1 mm, and preferably less than 600 microns.

FIG. 5C illustrates an example system 500 implementing the three-compartment carrier arm 510 configuration. In embodiments, the system 500 may include a handle 530 and a head 535 configured to support and orient the carrier arm 510. The handle 530 may be configured to facilitate positioning and insertion of the carrier arm 510 into the gingival crevice. The head 535 may be configured to couple the carrier arm 510 to the handle 530 and to maintain proper orientation during insertion.

In embodiments, after transphasic loading of medicament 540 into the compartments of the carrier arm 510, the system 500 may be used to advance the carrier arm 510 into the gingival crevice until the carrier arm 510 reaches the base of the gingival sulcus proximate to the junctional epithelium. The structural reinforcement provided by the first partitioner 516 and the second partitioner 517 may prevent deformation of the carrier arm 510 during insertion, preserving the geometric definition of the first compartment 520a, the second compartment 520b, and the third compartment 520c and ensuring controlled positioning of the medicament 540 at the base of the gingival crevice.

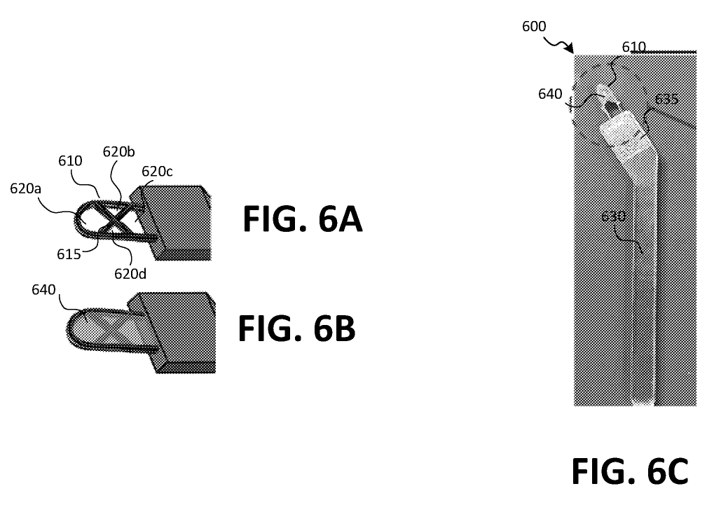

FIGS. 6A–6C illustrate embodiments of a four-compartment linear framework configuration implemented using a carrier arm 610 in accordance with embodiments of the present disclosure. In embodiments, the carrier arm 610 may be configured with an X-shaped partitioner 615 defining four distinct compartments within the medicament well.

As shown in FIG. 6A, the carrier arm 610 may be configured with a first compartment 620a, a second compartment 620b, a third compartment 620c, and a fourth compartment 620d. The first compartment 620a, the second compartment 620b, the third compartment 620c, and the fourth compartment 620d may be defined by an X partitioner 615 that extends across the interior region of the carrier arm 610. In embodiments, the X partitioner 615 may include two cross-members intersecting within the medicament well, subdividing the enclosed region into four compartments.

In embodiments, the X partitioner 615 may be formed integrally with the carrier arm 610 during manufacture. In embodiments, when the carrier arm 610 is formed from monofilament or braided filament, the X partitioner 615 may be formed by additional filament segments crossing within the planar geometry of the carrier arm 610. In embodiments, the intersection of the cross-members forming the X partitioner 615 may be bonded, fused, adhered, or otherwise structurally secured to maintain rigidity and geometric stability.

In embodiments, the X partitioner 615 may enhance structural integrity of the carrier arm 610. For example, the X partitioner 615 may reduce unsupported span length across the planar framework and may distribute mechanical forces across multiple structural paths during handling and insertion. In embodiments, without the X partitioner 615, the carrier arm 610 may deform during use, causing distortion of the medicament well geometry and potentially disrupting transphasic loading characteristics. By contrast, the X partitioner 615 may maintain dimensional stability of the first compartment 620a, the second compartment 620b, the third compartment 620c, and the fourth compartment 620d throughout loading, handling, and insertion into the gingival crevice.

FIG. 6B illustrates the carrier arm 610 after transphasic loading, in which medicament 640 may be retained within the first compartment 620a, the second compartment 620b, the third compartment 620c, and the fourth compartment 620d. The medicament 640 may represent the molded medicament seed formed after solvent dissipation. In some embodiments, the X partitioner 615 may preserve compartmental separation during and after loading, preventing undesired intermixing of medicament compositions where separation is intended.

In embodiments, the combined thickness of the carrier arm 610 and the medicament 640 retained within the four compartments may be configured to be less than 1 mm, and preferably less than 600 microns.

FIG. 6C illustrates an example system 600 implementing the four-compartment carrier arm 610 configuration. In embodiments, the system 600 may include a handle 630 and a head 635 configured to support and orient the carrier arm 610. The handle 630 may be configured to facilitate positioning and insertion of the carrier arm 610 into the gingival crevice. The head 635 may be configured to couple the carrier arm 610 to the handle 630 in a manner that maintains proper angular orientation relative to the gingival crevice during insertion.

In embodiments, after transphasic loading of medicament 640 into the compartments of the carrier arm 610, the system 600 may be used to advance the carrier arm 610 into the gingival crevice until the carrier arm 610 reaches the base of the gingival sulcus proximate to the junctional epithelium. The structural reinforcement provided by the X partitioner 615 may prevent deformation during insertion, preserving the geometric definition of the first compartment 620a, the second compartment 620b, the third compartment 620c, and the fourth compartment 620d and ensuring consistent placement of medicament 640 at the base of the gingival crevice.

In embodiments, the carrier frame may be configured as a semi-rigid carrier frame. When using a semi-rigid carrier frame, certain structural and geometric considerations may apply. For example, unlike monofilament, which may have shape limitations based on filament rigidity and bend radius, a semi-rigid carrier frame may allow greater flexibility in defining internal geometries and compartment layouts. The semi-rigid carrier frame may be formed from a polymer or composite material that may be configured to maintain dimensional stability during handling and insertion while allowing more complex internal patterns than may be achievable using bent filament alone.

In embodiments, the geometry of a semi-rigid carrier frame may be formed using a molding process, stamping process, extrusion process, additive manufacturing process, laser cutting process, and/or other manufacturing technique. For example, a polymer sheet or polymer preform may be stamped to define internal partitioners and compartment boundaries. In embodiments, injection molding may be used to create a carrier frame having defined internal walls that form compartments or sub-wells. In embodiments, because the semi-rigid carrier frame may be manufactured as a unified molded structure, the resulting geometry may include more intricate internal patterns such as parallel channels, diagonal partitions, cross-hatched meshes, honeycomb arrays, or other compartmental configurations.

In embodiments, semi-rigid carrier frames may include a three-dimensional depth component. For example, the carrier frame may include depth up to approximately 700 microns, within which defined wells or compartments may be formed and subsequently filled with medicament during transphasic loading. This depth may allow volumetric sub-wells to be created rather than purely planar enclosures. In embodiments, the depth dimension may influence medicament volume capacity, release kinetics, and structural rigidity, while still being configured to maintain an overall combined thickness compatible with insertion to the base of the gingival crevice proximate to the junctional epithelium.

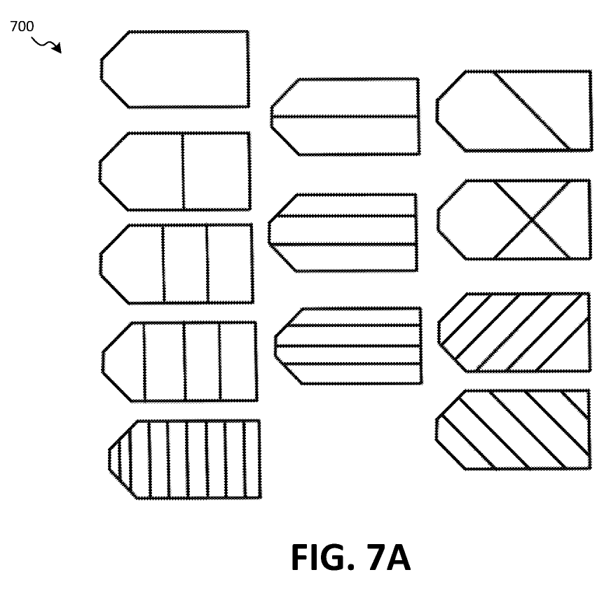

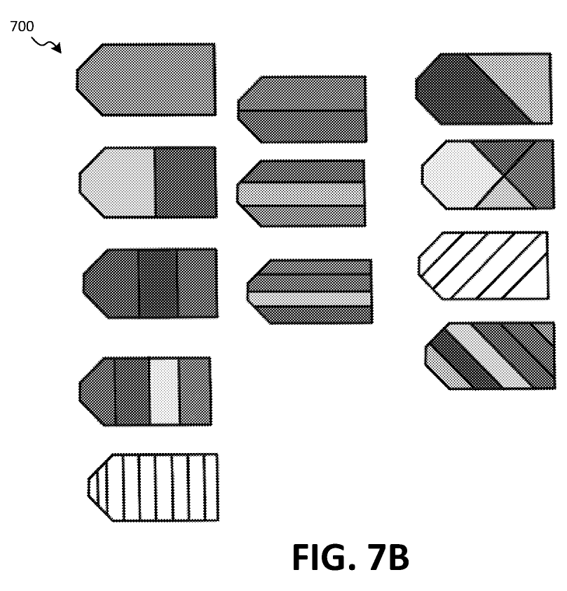

FIG. 7A illustrates various example geometries of a semi-rigid carrier frame 700 configured in accordance with embodiments of the present disclosure. As shown, the carrier frame 700 may be configured in multiple planar and/or shallow three-dimensional geometries. These geometries may include, without limitation, linear partitions, parallel partitions, diagonal partitions, X-shaped partitions, mesh-like configurations, honeycomb arrays, polygon arrays, cross-hatched structures, or combinations thereof. Each configuration may define one or more compartments or sub-wells within the carrier frame 700.

In embodiments, the geometry of the carrier frame 700 may define a single large medicament well, or may define multiple compartments arranged in series, parallel, radial, diagonal, or grid-based layouts. The number, size, and orientation of compartments may be selected based on particular medicament delivery requirements. For example, compartments arranged longitudinally may facilitate staged release along an insertion axis. Compartments arranged in parallel channels may provide layered dissolution behavior. Honeycomb, polygon, or mesh configurations may provide distributed release across a broader contact surface area within the gingival crevice.

In embodiments, the geometry of the carrier frame 700 may operate to define medicament delivery phases. For example, compartments located more apically may be configured with faster dissolving medicament compositions to initiate rapid delivery proximate to the junctional epithelium. Compartments located more coronally may be configured with slower dissolving compositions to provide sustained delivery over time. In embodiments, partition thickness, compartment depth, and compartment width may influence dissolution rates and release sequencing.

FIG. 7B illustrates the example geometries of the carrier frame 700 after loading with medicament in accordance with embodiments of the present disclosure. In embodiments, each compartment defined within the carrier frame 700 may be independently filled using transphasic molding techniques as described herein. The fillings within the compartments may be identical across compartments or may differ between compartments.

In embodiments, the compartments may be filled with pure drug, pure drug combined with excipients, drug encapsulated within nanoparticles, drug dispersed within polymer matrices, or combinations thereof. For example, one compartment may contain a pure drug formulation, while another compartment may contain drug encapsulated within nanoparticles configured for delayed release. In embodiments, one compartment may contain a hydrophilic formulation while another compartment may contain a hydrophobic formulation. In embodiments, some compartments may contain identical material while others contain distinct materials to enable multi-modal therapeutic delivery.

In embodiments, after solvent dissipation during transphasic loading, the medicament retained within each compartment may form a structured medicament seed integrated within the geometry of the carrier frame 700. The internal partitions of the carrier frame 700 may maintain separation between compartments where distinct compositions are used, preserving spatial control of release behavior within the gingival crevice.

FIG. 2D illustrates an example of a carrier frame 250 that is configured with a three-dimensional geometry defining a depth 255 in accordance with embodiments of the present disclosure. In embodiments, the carrier frame 250 shown in FIG. 2D may include a volumetric structure having measurable three-dimensional depth 255 extending orthogonally relative to a primary insertion plane.

In embodiments, the carrier frame 250 may be formed into a distinctive shape having defined sidewalls and a base such that a volumetric well is created. The depth 255 of the carrier frame 250 may be configured to be up to approximately 600 microns, approximately 700 microns, and in some embodiments up to 1 mm. In embodiments, the depth 255 may define a volumetric cavity capable of receiving and retaining a greater volume of medicament relative to planar linear configurations. In this manner, the carrier frame 250 may be configured to create a deeper well capable of holding increased medicament load during transphasic loading.

In embodiments, when the carrier frame 250 includes depth 255, the medicament solution applied during transphasic molding may wet and fill the volumetric well defined by the carrier frame 250. During the initial phase, surface tension and viscosity characteristics of the medicament solution may cause the medicament solution to conform to the geometry defined by the interior surfaces of the carrier frame 250. As solvent dissipates, the medicament may transition into a more solid or semi-solid phase while remaining retained within the volumetric well. Because of the increased depth 255, the molded medicament seed may have greater vertical thickness relative to planar configurations, increasing medicament capacity.

In embodiments, the carrier frame 250 may be configured in any geometrical shape. For example, the carrier frame 250 may be cylindrical, cubical, rectangular, polygonal, trapezoidal, or other three-dimensional geometric configuration. In embodiments, the sidewalls of the carrier frame 250 may be perpendicular, angled, curved, stepped, or otherwise configured to influence medicament retention and release characteristics. The geometry of the carrier frame 250 may be selected based on desired medicament volume, structural rigidity, release kinetics, or insertion characteristics.

In embodiments, carrier frames without three-dimensional depth (e.g., planar linear frameworks) may have structural thickness between approximately 10 microns and 70 microns, upon which surface coatings may be applied. In embodiments, carrier frames having three-dimensional depth 255 may have structural depths up to approximately 700 microns within which medicament wells may be formed and filled. The increased depth may allow compartmentalization within the volumetric well and may enable greater flexibility in configuring release phases and medicament distribution.

In embodiments, the composition of the carrier frame 250 may be similar to the compositions described herein for other carrier frame embodiments. For example, the carrier frame 250 may be formed from a biocompatible polymer, biodegradable polymer, composite material, plastic material, metal material, or combinations thereof. In embodiments, the carrier frame 250 may be formed from a dissolvable material configured to dissolve within the gingival crevice after deposition, allowing the carrier frame 250 to remain in situ along with the medicament seed. In embodiments, the carrier frame 250 may be non-dissolvable and may require removal after medicament release. In embodiments, the carrier frame 250 may be infused with medicament within its material matrix in addition to the medicament retained within the volumetric well.

In embodiments, although the depth 255 may be up to approximately 700 microns, the overall combined thickness of the carrier frame 250 and the medicament retained therewithin may still be configured to permit insertion to the base of the gingival sulcus proximate to the junctional epithelium. In embodiments, the dimensional configuration of the carrier frame 250 may be selected to balance increased medicament capacity with atraumatic insertion characteristics.

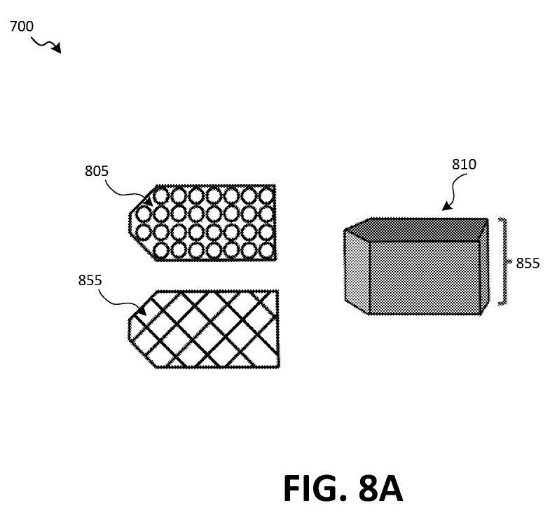

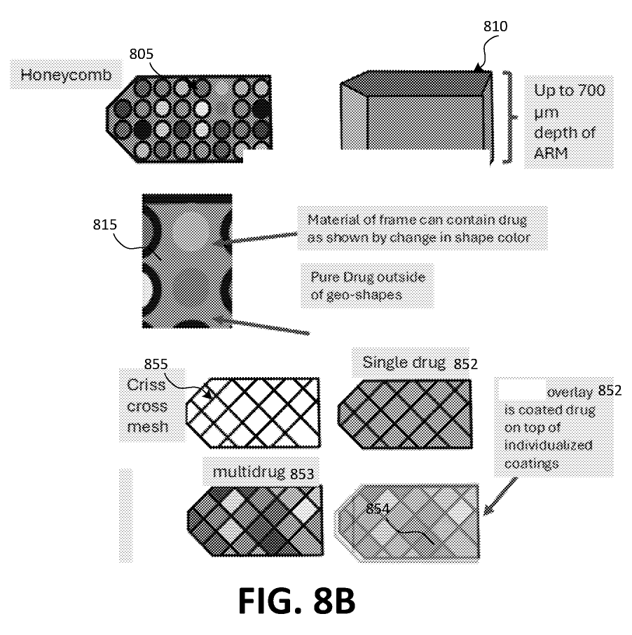

FIG. 8A illustrates examples of a semi-rigid carrier arm 810 configured with defined three-dimensional depth 855 and internal patterned geometries in accordance with embodiments of the present disclosure. In embodiments, the carrier arm 810 may include a volumetric structure having depth 855 extending orthogonally relative to a primary insertion plane. In embodiments, the depth 855 may be configured up to approximately 700 microns, as described herein.

In embodiments, the carrier arm 810 may include internal geometric patterns formed within the volumetric structure. In embodiments, one example pattern may include a honeycomb pattern 805. The honeycomb pattern 805 may define a plurality of closely packed sub-wells or micro-compartments distributed throughout the carrier arm 810. In embodiments, the honeycomb pattern 805 may increase surface area available for medicament loading and may provide structural rigidity due to the repeating cell structure.

In embodiments, another example pattern may include a criss-cross mesh pattern 855. The criss-cross mesh pattern 855 may define intersecting linear partitioners forming a grid-like structure within the carrier arm 810. In embodiments, the criss-cross mesh pattern 855 may create multiple interconnected sub-wells that may be filled during transphasic loading. The mesh configuration may provide enhanced mechanical strength and may resist deformation during insertion into the gingival crevice.

In embodiments, the honeycomb pattern 805 and the criss-cross mesh pattern 855 may represent example geometries only, and the carrier arm 810 may be configured with any array of internal geometric shapes. The internal geometry may be selected based on desired medicament capacity, release kinetics, mechanical rigidity, or manufacturing considerations. In embodiments, the depth 855 of the carrier arm 810 may allow volumetric wells within the honeycomb pattern 805 or criss-cross mesh pattern 855 to be filled with medicament during transphasic molding.

FIG. 8B further illustrates an example of the carrier arm 810 after loading with medicament in accordance with embodiments of the present disclosure. In embodiments, the material forming the carrier arm 810 may itself contain medicament., as shown in 815. For example, the polymer or semi-rigid material forming the structural frame may be infused with drug dispersed within its matrix, which is visually represented in FIG. 8B by variation in color within the geometric structure of the carrier arm 810. In such embodiments, medicament may be delivered both from the volumetric wells and from the structural frame material itself.

In embodiments, the compartments defined by the honeycomb pattern 805 or the criss-cross mesh pattern 855 may be filled with pure drug 852, or may be filled with pure drug combined with excipients, or drug encapsulated within nanoparticles dispersed within a carrier matrix. In embodiments, a single drug 852 may be loaded uniformly across all compartments of the carrier arm 810. In alternative embodiments, a multi-drug configuration 853 may be implemented in which different compartments within the honeycomb pattern 805 or criss-cross mesh pattern 855 contain different therapeutic agents.

In embodiments, an overlay 854 may be applied to at least a portion of the carrier arm 810 after compartmental loading. The overlay 854 may include coated drug layered on top of individualized compartment coatings. For example, the compartments may be initially loaded with one medicament composition and subsequently coated with a second medicament layer applied across the surface of the carrier arm 810. In embodiments, the overlay 854 may provide an initial rapid-release layer, while medicament within the compartments may provide delayed or sustained release following dissolution of the overlay 854.

In embodiments, the combined thickness of the carrier arm 810 and the medicament retained therewithin, as well as the overlay 852 when included, may remain less than 1mm, and preferably less than 600 microns, configured to permit insertion to the base of the gingival sulcus proximate to the junctional epithelium.

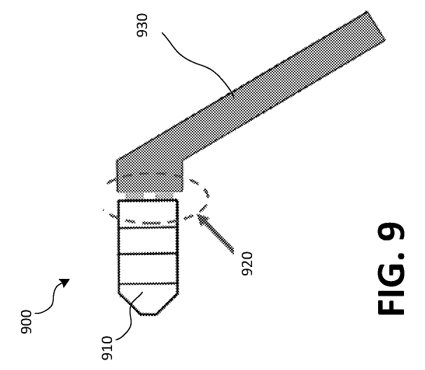

FIG. 9 illustrates an exemplary system 900 configured to provide releasable coupling between a carrier arm 910 and a handle 930 via a release interface 920 in accordance with embodiments of the present disclosure. In embodiments, the system 900 may include a carrier arm 910 configured to carry medicament for delivery into the gingival crevice, a handle 930 configured to facilitate positioning and insertion of the carrier arm 910, and a release interface 920 configured to releasably couple the carrier arm 910 to the handle 930.

In embodiments, configuring the carrier arm 910 with release points may include configuring release points between the carrier arm 910 and the handle 930 such that the handle 930 may be removably attached to the carrier arm 910 during insertion and detached after the carrier arm 910 has been positioned within the gingival crevice. The configuration and location of the release interface 920 may depend on the geometry and material properties of the carrier arm 910 and the handle 930. In embodiments, such as where the carrier arm 910 is semi-rigid, the release interface 920 may be positioned between a proximal region of the carrier arm 910 and a distal region of the handle 930.

In embodiments, the release interface 920 may include one or more inserts configured to be inserted into the carrier arm 910 and/or into the handle 930. These inserts may operate to releasably couple the carrier arm 910 to the handle 930. In embodiments, the release interface 920 may alternatively or additionally include tabs configured to attach the handle 930 to the carrier arm 910 and to break off upon application of a separation force. In this manner, the release interface 920 may enable the carrier arm 910 to be inserted into the gingival crevice using the handle 930, followed by detachment of the handle 930 from the carrier arm 910 by breaking or fracturing of the release interface 920.

In embodiments, the inserts of the release interface 920 may be formed from materials configured to crisply break when subjected to a threshold force. For example, the inserts may be formed from dry cellulose, wafer-like materials, brittle polymer compositions, or other materials having controlled fracture characteristics. In embodiments, the inserts may be configured in any geometric shape, may be oriented in any geometry relative to the carrier arm 910 and handle 930, and may be present in quantities ranging from a single insert to multiple inserts, for example between one and approximately one thousand inserts, and such as one, two, three, four, five, six, seven, eight, nine, or ten inserts, or between one and 20 inserts. In embodiments, the inserts may have dimensions between approximately 100 microns and 1000 microns. The number, orientation, and dimensions of the inserts may be selected to control the force required to detach the handle 930 from the carrier arm 910.

In embodiments, after insertion of the carrier arm 910 into the gingival crevice and positioning proximate to the junctional epithelium, the handle 930 may be detached by applying a force sufficient to fracture or disengage the release interface 920. In embodiments, after detachment, the handle 930 may be disposed of, reused with a new carrier arm 910 configured with new inserts, or otherwise reconditioned. In embodiments, the carrier arm 910 may remain within the gingival crevice for medicament delivery while the handle 930 is separated therefrom.

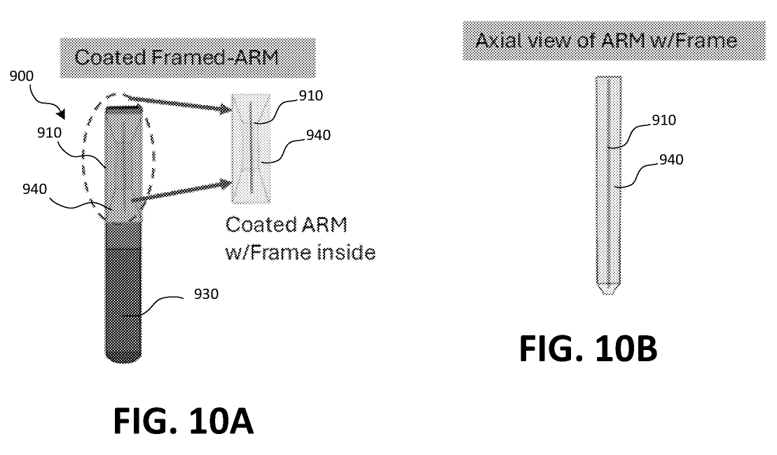

FIGS. 10A and 10B illustrate examples of a coated carrier arm 910 configured in accordance with embodiments of the present disclosure. As shown in FIG. 10A, the carrier arm 910 may be coupled to the handle 930 and coated with medicament 940. The medicament 940 may be applied onto the carrier arm 910 using transphasic loading techniques described herein.

In embodiments, the medicament 940 may coat the exterior surfaces of the carrier arm 910 and/or may reside within defined compartments of the carrier arm 910 where such compartments are present. In embodiments, the carrier arm 910 may be infused with medicament within its material matrix in addition to the medicament 940 coated on its surface. The configuration shown in FIG. 10A may represent a coated framed-arm configuration in which the carrier arm 910 carries medicament 940 both internally and externally. FIG. 10B illustrates an axial view of the carrier arm 910 with medicament 940. As shown in FIG. 10B, the thickness profile of the carrier arm 910 and the medicament 940 coating may include a combined thickness of the carrier arm 910 and the medicament 940 that remains less than 1 mm, and preferably less than 600 microns, to permit insertion to the base of the gingival sulcus proximate to the junctional epithelium.

In embodiments, the coated carrier arm 910 shown in FIGS. 10A and 10B may be inserted into the gingival crevice using the handle 930, and the handle 930 may be detached via the release interface 920 described with respect to FIG. 9. The carrier arm 910, with medicament 940 retained thereon, may remain within the gingival crevice for a period sufficient to allow release of medicament for absorption through the junctional epithelium.

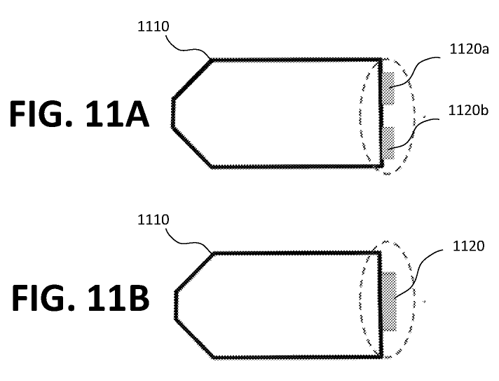

FIGS. 11A and 11B illustrate examples of a release interface configured to releasably couple a carrier frame 1110 to a handle in accordance with embodiments of the present disclosure. In embodiments, the carrier frame 1110 may be configured similarly to the carrier frames described herein and may define a medicament well configured to carry medicament for delivery into the gingival crevice. The embodiments shown in FIGS. 11A and 11B illustrate a configuration of releasable inserts or tabs configured to detach the carrier frame 1110 from the handle after insertion into the gingival crevice.

In embodiments, the release interface may include a plurality of releasable inserts 1120a and 1120b positioned at a proximal region of the carrier frame 1110. In embodiments, the releasable inserts 1120a and 1120b may be configured as square or rectangular tabs extending between the carrier frame 1110 and a corresponding receiving structure of the handle. The plurality of releasable inserts 1120a and 1120b may be configured to distribute mechanical load during insertion and may provide controlled breakage characteristics upon application of a detachment force.

In embodiments, the releasable inserts 1120a and 1120b may be formed from a material configured to crisply fracture when subjected to a threshold force. For example, the releasable inserts 1120a and 1120b may be formed from dry cellulose, wafer-like materials, brittle polymer compositions, or other materials selected for predictable fracture behavior. In embodiments, the thickness, width, and length of the releasable inserts 1120a and 1120b may be selected to control the force required to detach the carrier frame 1110 from the handle. In embodiments, the use of multiple releasable inserts 1120a and 1120b may allow fine-tuning of the break force by distributing stress across multiple break points.

FIG. 11B shows a release interface that includes a single releasable insert 1120 positioned between the carrier frame 1110 and the handle. In embodiments, the single releasable insert 1120 may be configured as a square or rectangular tab similar in geometry to releasable inserts 1120a and 1120b but implemented as a single structural element. In embodiments, the single releasable insert 1120 may simplify manufacturing and may provide a predictable single-point fracture mechanism for detaching the carrier frame 1110 from the handle.

In embodiments, the number of releasable inserts may vary based on desired detachment characteristics. For example, a single releasable insert 1120 may provide a lower break threshold and simplified geometry, while a plurality of releasable inserts 1120a and 1120b may provide increased structural stability during insertion and more distributed stress control prior to detachment. In embodiments, the releasable inserts may be oriented in any geometry relative to the carrier frame 1110 and the handle, and may be positioned symmetrically, asymmetrically, centrally, or peripherally depending on the mechanical design of the system.

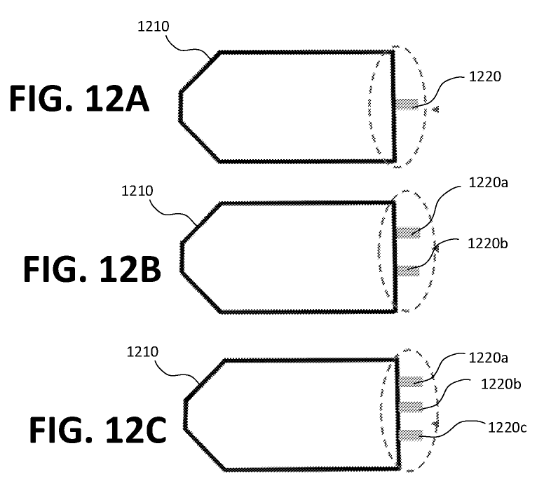

FIGS. 12A–12C illustrate examples of a release interface configured to releasably couple a carrier frame 1210 to a handle in accordance with embodiments of the present disclosure. In the embodiments illustrated in FIGS. 12A-12C, the releasable inserts may be cylindrical. In embodiments, the carrier frame 1210 may be configured similarly to the carrier frames described herein and may define a medicament well configured to carry medicament for delivery into the gingival crevice. The examples shown in FIGS. 12A–12C focus on cylindrical releasable inserts or tabs that are configured to detach the carrier frame 1210 from the handle after insertion into the gingival crevice.

In embodiments, the release interface may include a single cylindrical releasable insert 1220 positioned between the carrier frame 1210 and the handle. In embodiments, the cylindrical releasable insert 1220 may extend axially from a proximal region of the carrier frame 1210 and may be received within a corresponding aperture or socket of the handle. In embodiments, the cylindrical releasable insert 1220 may be configured to fracture, shear, or otherwise fail upon application of a threshold detachment force applied to the handle after insertion of the carrier frame 1210 into the gingival crevice.

FIG. 12B shows the carrier frame 1210 including a plurality of cylindrical releasable inserts 1220a and 1220b. In embodiments, the plurality of cylindrical releasable inserts 1220a and 1220b may provide increased mechanical stability during insertion of the carrier frame 1210 into the gingival crevice. The cylindrical releasable inserts 1220a and 1220b may distribute insertion forces across multiple structural elements while maintaining predictable fracture behavior during detachment. In embodiments, the diameters and lengths of cylindrical releasable inserts 1220a and 1220b may be selected to control the detachment force required to separate the carrier frame 1210 from the handle.

FIG. 12C shows an example of the carrier frame 1210 with three cylindrical releasable inserts 1220a, 1220b, and 1220c. In embodiments, the number of cylindrical releasable inserts may range from a single insert to multiple inserts depending on desired break strength, insertion stability, and manufacturing considerations. In embodiments, the cylindrical releasable inserts 1220a, 1220b, and 1220c may be arranged in parallel, symmetrically or asymmetrically relative to the longitudinal axis of the carrier frame 1210.

In embodiments, the cylindrical releasable inserts 1220, 1220a, 1220b, and 1220c may be formed from materials configured to provide controlled fracture characteristics. For example, the cylindrical releasable inserts may be formed from brittle polymers, dry cellulose, wafer-like materials, or other materials configured to crisply break or shear under applied force. In embodiments, the cylindrical releasable inserts may have diameters between approximately 100 microns and 1000 microns. In embodiments, the cross-sectional geometry, length, and material properties of the cylindrical releasable inserts may be selected to define a predictable separation threshold.

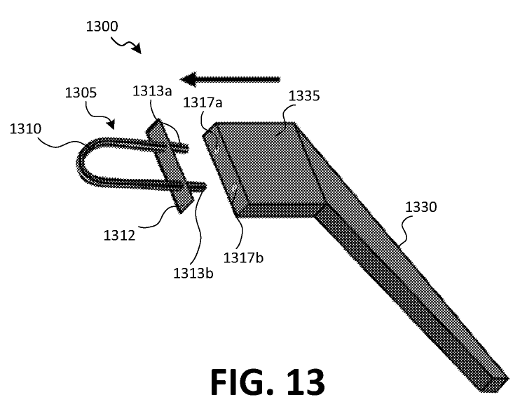

FIG. 13 illustrates an exemplary system 1300 in which the carrier is implemented as part of a carrier cartridge 1305 in accordance with embodiments of the present disclosure. In embodiments, the carrier cartridge 1305 may include a carrier arm 1310 configured to carry medicament for delivery into the gingival crevice, and a head 1335 configured to interface with a reusable handle 1330. In embodiments, the carrier cartridge 1305 may further include an optional structural component 1312 configured to provide reinforcement, alignment, or attachment functionality between the carrier arm 1310 and the head 1335.

In embodiments, the carrier arm 1310 may be configured in any of the geometries described herein, including planar linear frameworks, semi-rigid volumetric configurations, compartmentalized configurations, or combinations thereof. The carrier arm 1310 may be transphasically loaded with medicament prior to installation onto the handle 1330. In this manner, the carrier cartridge 1305 may represent a pre-loaded medicament delivery module configured to be coupled to the reusable handle 1330.

As shown in FIG. 13, the carrier cartridge 1305 may be configured to releasably couple to the handle 1330 via cartridge inserts 1313a and 1313b. The cartridge inserts 1313aand 1313bmay be received within corresponding apertures or receiving structures 1317a and 1317b defined within the head 1335 of the handle 1330. In embodiments, the cartridge inserts 1313aand 1313b may be configured as breakable inserts, non-breakable inserts, snap-fit inserts, friction-fit inserts, or other releasable coupling mechanisms.

In embodiments, the cartridge inserts 1313a and 1313b may be configured to securely attach the carrier cartridge 1305 to the handle 1330 during insertion of the carrier arm 1310 into the gingival crevice. After deposition of the medicament into the gingival crevice, the carrier cartridge 1305 may be released from the handle 1330. For example, the carrier arm 1310 may be inserted into the gingival crevice until it reaches the base of the gingival sulcus proximate to the junctional epithelium, and thereafter the handle 1330 may be detached from the carrier cartridge 1305 by disengaging the cartridge inserts 1313a and 1313b from the receiving structures 1317a and 1317b.

In embodiments, the carrier cartridge 1305 may be configured such that the carrier arm 1310 remains within the gingival crevice while the head 1335 and the handle 1330 are removed. In other embodiments, the carrier arm 1310 and the head 1335 may remain attached and may be removed together after sufficient time has elapsed for medicament delivery. In embodiments, the cartridge inserts 1313a and 1313b may be configured as breakable elements that fracture upon application of a threshold force, leaving at least a portion of the carrier cartridge 1305 within the gingival crevice while allowing the handle 1330 to be removed.

In embodiments, after removal of the carrier cartridge 1305 from the handle 1330, a new carrier cartridge 1305 including a new carrier arm 1310 pre-loaded with medicament may be installed onto the reusable handle 1330. In this manner, the system 1300 may be configured to allow repeated medicament applications using a single reusable handle 1330 and multiple disposable carrier cartridges 1305.

In embodiments, a hybrid configuration may be implemented in which the carrier arm 1310 is breakable relative to the carrier cartridge 1305. For example, the carrier arm 1310 may be broken off from the structural component 1312 or head 1335 and left within the gingival crevice, while the remainder of the carrier cartridge 1305 remains attached to the handle 1330. In these embodiments, a subsequent carrier cartridge 1305 may include a new carrier arm 1310 configured to be broken off in a similar manner.

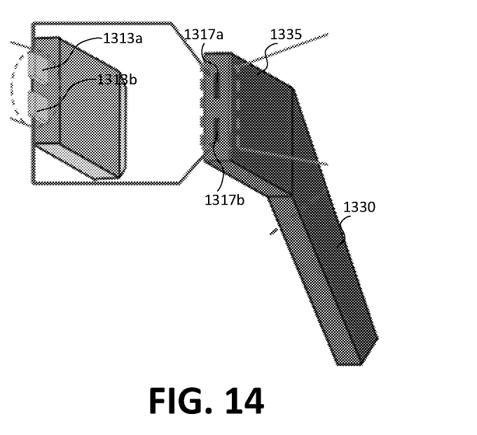

FIG. 14 illustrates an example of the cartridge-based release interface in which cartridge inserts are used to releasably attach a carrier cartridge to a reusable handle in accordance with embodiments of the present disclosure. As shown in FIG. 14, the carrier cartridge may include cartridge inserts 1313a and 1313b extending from the cartridge body toward corresponding receiving structures 1317a and 1317b formed within the head 1335 of the reusable handle 1330. In embodiments, the cartridge inserts 1313a and 1313b may be received within alignment casing holes defined within the head 1335. The alignment casing holes may guide insertion of the cartridge inserts 1313a and 1313b to ensure proper orientation of the carrier relative to the handle 1330 prior to insertion into the gingival crevice.

In embodiments, the cartridge inserts 1313a and 1313b may be configured as breakable inserts formed from materials such as cellulose-based materials, wafer-like materials, brittle polymers, or other materials configured to easily fracture under a defined threshold force. In embodiments, the cartridge inserts 1313a and 1313b may be dimensioned and oriented such that insertion forces encountered during positioning of the carrier within the gingival crevice are insufficient to cause breakage, while deliberate separation forces applied after positioning may cause fracture.

In embodiments, during use, the carrier cartridge may be inserted into the reusable handle 1330 by engaging the cartridge inserts 1313a and 1313b within the receiving structures 1317a and 1317b of the head 1335. The handle 1330 may then be used to insert the carrier into the gingival crevice until the carrier reaches the base of the gingival sulcus proximate to the junctional epithelium.

In embodiments, after deposition of the medicament into the gingival crevice, the user may apply a detachment force to the handle 1330. In one configuration, the cartridge inserts 1313a and 1313b may break, allowing the carrier portion of the cartridge to remain within the gingival crevice while the head 1335 and the reusable handle 1330 are removed. In another configuration, the entire cartridge may remain intact and may be removed from the gingival crevice after sufficient time has elapsed for medicament release, and thereafter the entire cartridge may be disengaged from the handle 1330 and replaced with a new carrier cartridge.

In embodiments, the reusable handle 1330 may be configured for repeated use with successive carrier cartridges. For example, after removal of a used carrier cartridge, a new carrier cartridge including a new carrier and medicament seed may be inserted into the receiving structures 1317a and 1317b of the head 1335. In embodiments, the alignment casing holes may ensure that the new cartridge is properly positioned relative to the insertion axis of the handle 1330.

In embodiments, the cartridge may also be configured such that only the carrier portion breaks off while the structural body of the cartridge remains attached to the handle 1330. In these embodiments, a subsequent cartridge may include a new breakable carrier section configured to detach in a similar manner. In this way, a combination of breakable inserts and reloadable cartridge structures may be used to enable selective in situ deposition of the carrier while preserving the reusable functionality of the handle 1330.

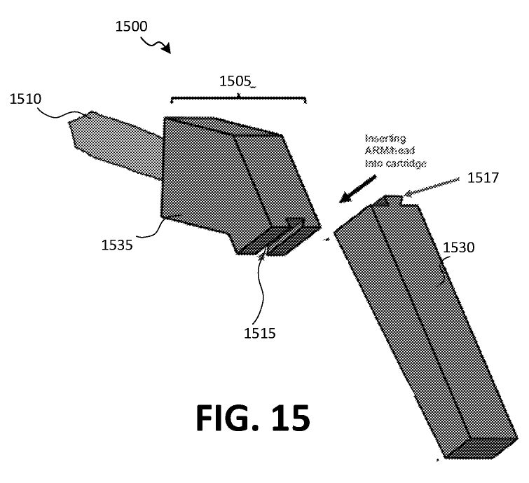

FIG. 15 illustrates an exemplary system 1500 in which the head of the carrier cartridge is configured as part of a removable cartridge assembly that is attachable to and detachable from a reusable handle in accordance with embodiments of the present disclosure. In embodiments, the system 1500 may include a reusable handle 1530 and a carrier cartridge 1505. The reusable handle 1530 may include a handle body configured to accept and secure the carrier cartridge 1505.

In embodiments, a head 1535 may be configured to attach to the handle 1530. In some embodiments, the head 1535 may be part of the carrier cartridge 1505 itself and may be removable from the handle 1530. In the configuration illustrated in FIG. 15, the carrier cartridge 1505 may be configured to be coupled or attached to the reusable handle 1530 by engaging a guide 1517 with a corresponding channel 1515.

In embodiments, the channel 1515 and the guide 1517 may operate as cartridge inserts configured to attach the carrier cartridge 1505 to the reusable handle 1530. The channel 1515 may be defined within the head 1535 of the handle 1510 and the guide 1517 may be defined on the reusable handle 1530. In alternative embodiments, the channel 1515 may be defined on the reusable handle 1530 and the guide 1517 may be defined within the head 1535. In either configuration, the channel and guide interface may provide alignment and secure coupling between the carrier cartridge 1505 and the reusable handle 1530.

In embodiments, the channel 1515 and guide 1517 may be configured to provide friction-fit engagement, snap-fit engagement, interference-fit engagement, or other releasable coupling mechanism. The channel 1515 and guide 1517 may ensure that the carrier cartridge 1505 is properly oriented relative to the insertion axis of the reusable handle 1530 prior to insertion of the carrier frame 1510 into the gingival crevice. In embodiments, the channel 1515 and guide 1517 may operate to distribute insertion forces and stabilize the carrier cartridge 1505 during positioning of the carrier arm within the gingival crevice.

In embodiments, after deposition of medicament within the gingival crevice, the carrier cartridge 1505 may be disengaged from the reusable handle 1530 by withdrawing the guide 1517 from the channel 1515. In embodiments, the entire carrier cartridge 1505 may be removed and replaced with a new carrier cartridge 1505 including a newly loaded carrier arm configured with medicament on the carrier frame 1510. In embodiments, the reusable handle 1530 may be used repeatedly with multiple disposable carrier cartridges.

In embodiments, the carrier cartridge 1505 may include a carrier frame 1510, and the carrier frame 1510 may be configured to either remain within the gingival crevice after breakage from the cartridge or be removed together with the cartridge after medicament delivery, depending on the release interface configuration described elsewhere herein.

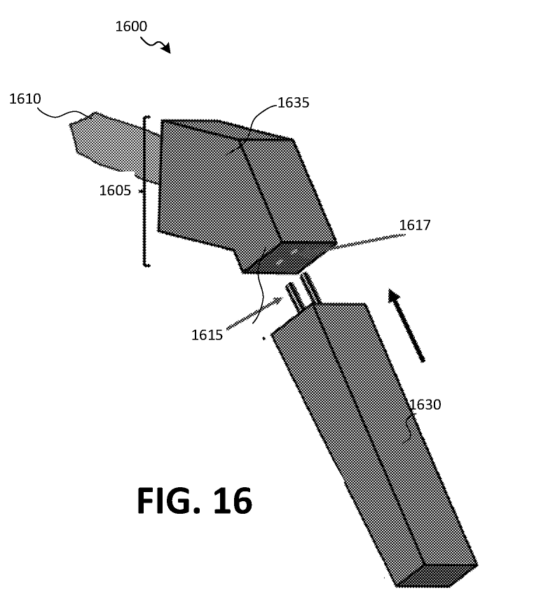

FIG. 16 illustrates an embodiment of a system 1600 in which the carrier cartridge includes a head that is removably attachable to a reusable handle using insertion rods and holes configured as cartridge inserts in accordance with embodiments of the present disclosure. In embodiments, the system 1600 may include a reusable handle 1630 and a carrier cartridge 1605. The reusable handle 1630 may include a handle body.

In embodiments, the head 1635 may be part of the carrier cartridge 1605 or may be part of the reusable handle 1630. In this configuration, the entire carrier cartridge 1605, including the head 1635 and the carrier frame 1610 integrated therewith, may be removably coupled to the reusable handle 1630. In embodiments, the head 1635 of the carrier cartridge 1605 may define one or more holes 1617 configured to receive corresponding insertion rods 1615 extending from the reusable handle 1630. In alternative embodiments, the holes 1617 may be defined within the reusable handle 1630 and the insertion rods 1617 may extend from the head 1635 of the carrier cartridge 1605.

In embodiments, the insertion rods 1615 and the holes 1617 may operate as cartridge inserts configured to provide alignment and releasable coupling between the carrier cartridge 1605 and the reusable handle 1630. The insertion rods 1615 may be dimensioned to fit within the holes 1617 using a friction-fit, interference-fit, snap-fit, or other releasable engagement mechanism. In embodiments, the number, spacing, and orientation of the insertion rods 1615 and holes 1617 may be selected to provide stable attachment during insertion of the carrier arm into the gingival crevice.

In embodiments, during operation, the carrier cartridge 1605 may be attached to the reusable handle 1630 by aligning the insertion rods 1615 with the holes 1617 and advancing the carrier cartridge 1605 into engagement. Once coupled, the reusable handle 1630 may be used to position and insert the carrier arm portion of the carrier cartridge 1605 into the gingival crevice until the carrier frame 1610 reaches the base of the gingival sulcus proximate to the junctional epithelium.

In embodiments, after medicament deposition within the gingival crevice, the carrier cartridge 1605 may be disengaged from the reusable handle 1630 by withdrawing the insertion rods 1615 from the holes 1617. In embodiments, the entire carrier cartridge 1605 may be removed from the gingival crevice and replaced with a new carrier cartridge 1605 pre-loaded with medicament. In alternative embodiments, the carrier frame portion of the carrier cartridge 1605 may be configured to detach or break off within the gingival crevice while the head 1635 remains coupled to the reusable handle 1630, depending on the release interface configuration described herein.

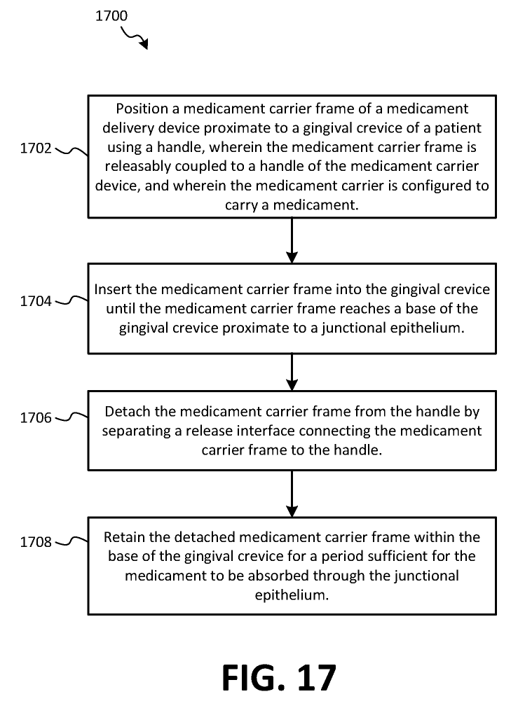

Operations for targeting delivery of a medicament to the junctional epithelium of a gingival crevice of a patient using a medicament delivery device configured in accordance with embodiments of the present disclosure will now be discussed with respect to FIG. 17. FIG. 17 shows a high-level flow diagram 1700 of operations for a medicament delivery device including a releasable carrier frame configured to carry a medicament seed and including one or more releasable points enabling deposition within the gingival crevice for absorption through the junctional epithelium in accordance with embodiments of the present disclosure. In embodiments, the operations of flow diagram 1700 may include operations for delivery of the releasable carrier frame configured as a medicament carrier cartridge disclosed herein and as described with respect to preceding figures.

At block 1702, a medicament carrier frame of a medicament delivery device may be positioned proximate to the gingival crevice of a patient using a handle. In embodiments, the handle may be configured to operate as the user interface for positioning and deploying the medicament carrier frame into the gingival crevice of the patient. In embodiments, the medicament carrier frame may be configured to carry a medicament and may be releasably coupled to the handle via a release interface. In some embodiments, the handle may be pre-loaded with a medicament carrier cartridge, while in other embodiments, a user may attach a new carrier cartridge to a reusable handle prior to positioning.

At block 1704, the medicament carrier frame of the medicament delivery device may be inserted into the gingival crevice of the patient. In embodiments, the insertion may include utilizing the handle device to advance the medicament carrier frame deep into the gingival crevice until it reaches a base of the gingival crevice proximate to a junctional epithelium. In embodiments, the combined width of the medicament carrier frame and the medicament retained therewithin may be configured to be less than 1 mm, or preferably less than 600 microns, to facilitate reaching the base of the gingival crevice proximate to the junctional epithelium.

At block 1706, the medicament carrier frame is detached from the handle. In embodiments, the detachment may include separating the release interface connecting the medicament carrier frame to the handle. In some embodiments, the separation may include applying a force to crisply break off one or more breakable inserts of the release interface, such as dry cellulose or wafer-like tabs, to release the medicament carrier frame from the handle. In other embodiments, the separation may include manipulating the handle to disengage an alignment guide from an insertion channel configured as a friction fit or interference fit. In embodiments, the detachment may leave the medicament carrier frame and the medicament deposited within the base of the gingival crevice while the handle is removed from the oral cavity.

At block 1708, the detached medicament frame is retained within the base of the gingival crevice for a period sufficient for the medicament to be absorbed through the junctional epithelium. In embodiments, the deposited medicament carrier frame maintains the medicament in close proximity to the junctional epithelium to facilitate absorption of the medicament into the patient’s system. In embodiments, while retained, the medicament carrier frame of the medicament carrier may dissolve within the gingival crevice along with the medicament seed. In other embodiments, the structural frame may be non-dissolvable and may be configured to be removed from the gingival crevice at a later time after the medicament payload has been released.

Although the present disclosure and its advantages have been described in detail, it should be understood that various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

Moreover, the description in this patent document should not be read as implying that any particular element, step, or function can be an essential or critical element that must be included in the claim scope. Also, none of the claims can be intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” “processing device,” or “controller” within a claim can be understood and intended to refer to structures known to those skilled in the relevant art, as further modified or enhanced by the features of the claims themselves, and can be not intended to invoke 35 U.S.C. § 112(f). Even under the broadest reasonable interpretation, in light of this paragraph of this specification, the claims are not intended to invoke 35 U.S.C. § 112(f) absent the specific language described above.

The disclosure may be embodied in other specific forms without departing from the spirit or essential characteristics thereof. For example, each of the new structures described herein, may be modified to suit particular local variations or requirements while retaining their basic configurations or structural relationships with each other or while performing the same or similar functions described herein. The present embodiments are therefore to be considered in all respects as illustrative and not restrictive. Accordingly, the scope of the disclosures can be established by the appended claims rather than by the foregoing description. All changes which come within the meaning and range of equivalency of the claims are therefore intended to be embraced therein. Further, the individual elements of the claims are not well-understood, routine, or conventional. Instead, the claims are directed to the unconventional inventive concept described in the specification.

What is claimed is:

1. A medicament carrier cartridge configured for delivering a therapeutic agent into a junctional epithelium of a gingival crevice of a patient, comprising:
a structural frame defining a geometric well having a defined depth, the geometric well configured to carry a medicament molded therewithin; and
a release interface integrated with the structural frame, wherein the release interface includes one or more release inserts configured to releasably couple the structural frame to a handle device and to facilitate the detachment of the structural frame from the handle device for deposition within the gingival crevice.

2. The medicament carrier cartridge of claim 1, wherein the structural frame is configured with a combined width that includes a width of the structural frame and a width of the medicament retained therewithin, wherein the combined width is less than 1 mm to allow the structural frame to reach a base of the gingival crevice proximate to the junctional epithelium to enable the medicament to be absorbed through the junctional epithelium.

3. The medicament carrier cartridge of claim 2, wherein the combined width is less than 600 microns.

4. The medicament carrier cartridge of claim 1, wherein the structural frame includes a filament including one of a monofilament having a thickness between 25 and 600 microns and a braided filament.

5. The medicament carrier cartridge of claim 1, wherein the structural frame includes a semi-rigid polymer formed by a mold or stamping process.

6. The medicament carrier cartridge of claim 1, wherein the geometric well includes a plurality of compartments separated by one or more partitioner, wherein the partitioners provide structural support for the structural frame and define distinct zones for medicament loading.

7. The medicament carrier cartridge of claim 6, wherein different compartments of the plurality of compartments are configured to one or more of:
carry different medicaments; and
release medicament at different phases.

8. The medicament carrier cartridge of claim 1, wherein the medicament retained within the geometric well includes a solution including one or more of a surfactant and a viscosity enhancer configured to control a surface tension to enable the solution to attain a shape of the geometric well during molding.

9. The medicament carrier cartridge of claim 1, wherein the one or more release inserts include a breakable material including one or more of dry cellulose and a wafer-like material, configured to crisply break the structural frame free from the handle device.

10. The medicament carrier cartridge of claim 1, wherein the structural frame includes a dissolvable material configured to dissolve within the gingival crevice along with the medicament.

11. A system for delivering medicament into a junctional epithelium of a gingival crevice of a patient, the system comprising:
a handle device configured to facilitate positioning within an oral cavity of the patient;
a medicament carrier including a frame structure carrying a medicament seed and a connection feature configured to interface with the handle device; and
a coupling mechanism formed between the handle device and the medicament carrier, wherein the coupling mechanism includes one or more detachable release points configured to mechanically connect the medicament carrier to the handle device during insertion and to separate the medicament carrier from the handle device upon deposition of the medicament seed into the gingival crevice for absorption of the medicament through the junctional epithelium.

12. The system of claim 11, wherein the medicament carrier is configured to have a combined width, including the frame structure and the medicament seed, of less than 600 microns, enabling the frame structure to reach a base of the gingival crevice proximate to the junctional epithelium.

13. The system of claim 11, wherein the one or more detachable release points include breakable inserts formed of a material different from the handle device, wherein the breakable inserts are configured to fracture upon application of a force to detach the medicament carrier from the handle device.

14. The system of claim 13, wherein the breakable inserts comprise a dry cellulose or wafer-like material.

15. The system of claim 11, wherein the connection feature of the medicament carrier includes an alignment guide and the handle device includes a corresponding insertion channel, the alignment guide and insertion channel configured to mate to form the coupling mechanism.

16. The system of claim 15, wherein the coupling mechanism is a friction fit or interference fit that allows the medicament carrier to be pulled off the handle device after deposition.

17. The system of claim 11, wherein the medicament carrier is a replaceable cartridge, and wherein the handle device is reusable and configured to receive a plurality of medicament carriers sequentially.

18. The system of claim 11, wherein the frame structure of the medicament carrier defines a well having a depth of up to 600 microns.

19. The system of claim 11, wherein the frame structure includes a semi-rigid polymer mesh or polygon structure configured to hold the medicament seed.

20. The system of claim 11, wherein the medicament carrier is dissolvable within the gingival crevice such that separation from the handle device leaves the entire medicament carrier within a base of the gingival crevice for absorption of the medicament through the junctional epithelium via dissolution of the medicament.
